(19) United States
(12) Patent Application Publication
(10) Pub. No.: US 2024/0360182 A1
(43) Pub. Date: Oct. 31, 2024

(54) Foot and Mouth Disease Virus (FMDV) Consensus Proteins, Coding Sequences Therefor and Vaccines Made Therefrom

(71) Applicants: The Trustees of the University of Pennsylvania, Philadelphia, PA (US); Inovio Pharmaceuticals, Inc., Plymouth Meeting, PA (US)

(72) Inventors: David B. Weiner, Merion, PA (US); Bernadette Ferraro, La Jolla, CA (US); Jian Yan, Wallingford, PA (US); Patricia A. Brown, Magnolia, TX (US); Rodney A. Bowling, Austin, TX (US); Douglas R. Kern, The Woodlands, TX (US); Mathura P. Ramanathan, Ardmore, PA (US); Niranjan Y. Sardesai, Blue Bell, PA (US); Karuppiah Muthumani, Cherry Hill, NJ (US)

(21) Appl. No.: 18/768,701

(22) Filed: Jul. 10, 2024

Related U.S. Application Data
(63) Continuation of application No. 17/474,732, filed on Sep. 14, 2021, now Pat. No. 12,049,479.
Application No. 17/474,732 is a continuation of application No. 16/416,663, filed on May 20, 2019, now Pat. No. 11,124,545.
Application No. 16/416,663 is a continuation of application No. 14/816,120, filed on Aug. 3, 2015, now Pat. No. 10,294,278.
Application No. 14/816,120 is a division of application No. 13/503,828, filed on Jul. 9, 2012, now Pat. No. 9,109,014.
(60) Provisional application No. 61/257,450, filed on Nov. 2, 2009.
(60) Provisional application No. 61/257,461, filed on Nov. 2, 2009.

(51) Int. Cl. C07K 14/005; A61K 39/00; A61K 39/12; A61K 39/135; A61K 39/39; A61M 37/00; A61N 1/32; C12N 7/00; G01N 33/569

(52) U.S. Cl. CPC C07K 14/005; A61K 39/12; A61K 39/135; A61K 39/39; A61M 37/00; A61N 1/327; C12N 7/00; G01N 33/56983; A61K 2039/523; A61K 2039/53; A61K 2039/552; C12N 2770/32122; C12N 2770/32134; G01N 2333/09; G01N 2469/20

(57) ABSTRACT

Provided herein is a nucleic acid comprising consensus amino acid sequence of foot-and-mouth disease FMDV VP1-4 coat proteins of FMDV subtypes A, Asia 1, C, O, SAT1, SAT2, and SAT3 as well as plasmids and vaccines expressing the sequences. Also provided herein is methods for generating an immune response against one or more FMDV subtypes using the vaccine as described above as well as methods for deciphering between vaccinated mammals with the vaccine and those that are infected with FMDV.

### FIELD OF THE INVENTION

The present invention relates to synthetic, consensus foot-and-mouth disease virus (FMDV) immunogenic proteins and nucleic acid molecule encoding such proteins, to vaccines against FMDV, to methods for inducing immune responses against FMVD, to methods for distinguishing between individuals infected with FMDV versus those vaccinated against FMDV, and methods of prophylactically and/or therapeutically immunizing individuals against FMDV.

### BACKGROUND OF THE INVENTION

Foot-and-mouth disease is a highly contagious disease of domestic and wild cloven-hoofed animals including cattle, swine, goats and deer which rapidly replicates in the host and spreads to in-contact susceptible animals. The disease is characterized by fever, lameness, and vesicular lesions of the tongue, feet, snout, and teats resulting in high morbidity, but low mortality in adult animals. The causative agent is the foot-and-mouth disease virus (FMDV), the type of species of the Aphthovirus genus, of the Picornaviridae family. FMDV is a single-stranded, positive-sense RNA genome of approximately 8500 bases surrounded by an icosahedral capsid with 60 copies each of four structural proteins VP1-4 and is antigenically highly variable with several subtypes including A, Asia 1, O, C, SAT1, SAT2, and SAT3. Recent outbreaks of foot-and-mouth disease in a number of previously disease free countries including Taiwan in 1997, United Kingdom and Netherlands in 2001, and the emergence in several South American countries has risen the awareness of the economically destructive virus. Furthermore, there is world-wide concern that a possible terrorist attack may target countries such as the US $100 billion/year livestock industry by employing FMDV.

Previous measures to control FMDV include slaughter of the infected or in-contact animals and decontamination. Countries that slaughter their livestock due to a FMDV outbreak can only resume livestock activities if the countries have FMDV free status for 3 months after the last outbreak. Countries usually use vaccination of the animals to treat an FMDV outbreak as a last resort because countries that have vaccinated and do not slaughter the animals must wait an entire year to regain FMD free status. Countries however are looking to vaccinate their animals before any FMDV outbreak and would be able to retain their FMD free status.

In the past, FMDV vaccines included chemically inactivated whole virus antigen in conjunction with an adjuvant; however, there are disadvantages to this because it requires expensive high-containment manufacturing facilities to produce the vaccine. Over the past 25-30 years investigators have been trying to develop a vaccine that provides protection after a single inoculation. These efforts include the use of VP1 purified from virus particles, bioengineered VP1, VP1 peptides, chemically synthesized VP1 peptides, live vectors expressing VP1 epitopes, inoculation with DNA encoding VP1 epitopes, and using the full capsid protein VP1-4 produced from FMDV-infected cultures or delivery of the VP1-4 capsid via replication defective human adenovirus type 5 (Ad5) vector. All of these approaches present only a limited number of epitopes across all the subtypes of the FMDV viruses to the inoculated animal.

Accordingly, there is a need in the art for a vaccine and methods of diagnosing FMDV infected mammals that is suitable to provide protection against a plurality of epitopes of FMDV across the various subtypes of FDMV.

### SUMMARY OF THE INVENTION

Provided herein is an isolated nucleic acid comprising a sequence encoding the consensus amino acid sequence of VP1-4 of foot-and-mouth disease virus subtypes A, Asia 1, C, O, SAT1, SAT2, SAT3, SAT4, or a complement thereof. The nucleic acid may comprise a sequence selected from the group consisting of (a) SEQ ID NOS: 17-23; (b) a nucleotide sequence encoding the amino acid sequence of 24-30; (c) a 80% variant of (a); and a complement of (a) or (b). Also provides is a vector comprising a heterologous sequence wherein the heterologous sequence consists of the sequence described above.

Also provided herein is a vaccine capable of generating in a mammal an immune response against a plurality of foot-and-mouth disease virus (FMDV) subtypes where the vaccine comprises a DNA plasmid comprising a promoter operably linked to a coding sequence that encodes a consensus FMDV antigen comprising capsid proteins VP1-4 from one or more FMDV subtypes and a pharmaceutically acceptable excipient wherein the DNA plasmid is capable of expressing the consensus FMDV antigen in a cell of the mammal in a quantity effective to elicit an immune response in the mammal. The vaccine may generate an immune response against FMDV subtypes A, Asia 1, C, O, SAT1, SAT2, SAT3 or combinations thereof. The coding sequences of the plasmid of the vaccine may be of the FMDV antigen selected from the group consisting of SEQ ID NOS: 1-7 or combinations thereof. The coding sequences of the plasmid of the vaccine may further comprise a N terminal leader sequence wherein the leader sequence is IgG or IgE. The plasmid of the vaccine may further comprise a polyadenylation sequence following the 3′ end of the coding sequence. The plasmid of the vaccine may further comprise a nucleotide sequence encoding a consensus FMDV 3C protease from subtypes A, Asia 1, C, O, SAT1, SAT2, or SAT3. The nucleotide sequence of FMDV 3C protease may be SEQ ID NO: 15 and may encoded by the amino acid sequence as set forth in SEQ ID NO: 16. The plasmid of the vaccine may be codon optimized. The coding sequence of the FMDV antigen may also comprise VP1-4 and 3C protease including SEQ ID NOS: 7-14. The pharmaceutically acceptable excipient of the vaccine may be adjuvant and the adjuvant may be IL-2 or IL-15. The pharmaceutically acceptable excipient of the vaccine may be a transfection facilitating agent. The transfection facilitating agent may be a polyanion, polycation or lipid such as poly-L-glutamate at a concentration of less than 6 mg/ml. The vaccine may be administered to a swine, ruminant, human or a primate. The vaccine may elicit a humoral or cellular or both a humoral and cellular response.

Also provided herein is a vaccine capable of generating in a mammal an immune response against a plurality of foot-and-mouth disease virus (FMDV) subtypes where the vaccine comprises one or more DNA plasmids comprising a promoter operatively linked to a coding sequence that encodes a consensus FMDV antigen comprising capsid proteins VP1-4 from one or more FMDV subtypes selected from the group consisting of subtypes A, Asia 1, C, O, SAT1, SAT2, SAT3, or a combination thereof and a pharmaceutically acceptable excipient thereof wherein the DNA plasmids are capable of expressing a consensus FMDV antigen in a cell of the mammal in a quantity effective to elicit an immune response in the mammal. The coding sequence of the FMDV antigen may be selected from the group consisting of SEQ ID NOS: 1-7 or a combination thereof. The plasmid of the vaccine may also further comprise a nucleotide sequence encoding a consensus 3C protease of FMDV for subtypes A, Asia1, C, O. SAT1, SAT2, or SAT3 and may comprise the nucleotide sequences set forth in SEQ ID NO: 15. The vaccine may be administered to a mammal such as swine, ruminant, human or a primate. The vaccine may elicit an immune response in a mammal such as a humoral, cellular, or both a humoral and cellular response.

Also provided herein is a vaccine capable of generating in a mammal an immune response against a plurality of FDMV subtypes where the vaccine comprises an antigen comprising one or more consensus amino acid sequences encoding capsid proteins VP1-4 of foot-and-mouth-disease virus (FMDV) subtypes A, Asia 1, C, O, SAT1, SAT2, or SAT3 and a pharmaceutically acceptable excipient thereof. The coding amino acid sequence of the FMDV antigen may be SEQ ID NOS: 24-30. The pharmaceutically acceptable excipient may be an adjuvant selected from the group consisting of IL-2 and IL-15. The pharmaceutically acceptable excipient of the vaccine may be transfection facilitating agent. The transfection facilitating agent may be a polyanion, polycation or a lipid such as poly-L-glutamate at a concentration of less than 6 mg/ml. The vaccine may be administered to a mammal such as a swine, ruminant, human or primate. The vaccine may elicit an immune response in a mammal such as a humoral, cellular, or both a humoral and cellular response.

Also provided herein is a method for eliciting an immune response against a plurality of FMDV virus subtypes in a mammal comprising delivering the DNA plasmid vaccine of claim 1 or 21 to the tissue of the mammal and electroporating cells of the tissue with a pulse of energy at a constant current effective to permit entry of the DNA plasmid into the cells. The delivery of the DNA plasmid of claim 1 in the method may comprise injecting the DNA plasmid vaccine into the intradermic, subcutaneous, or muscle tissue. The DNA plasmid of the method may be delivered by presetting the current and the pulse of energy is at a constant current that equals the present current. The electroporation step of the method may further comprise measuring the impedence in the electroporated cells, adjusting the energy level of the pulse of energy relative to the measured impedence to maintain a constant current in the electroporated cells wherein the measuring and adjusting step occurs within a lifetime of the pulse of energy. The electroporating step may further comprise delivering the pulse of energy to a plurality of electrodes according to a pulse sequence pattern that delivers the pulse of energy in a decentralized pattern.

Also provided is a method of diagnosing a mammals infected with FMDV wherein the method comprises isolating a fluid sample from the mammal, isolating antibodies from the fluid sample of the mammal, and comparing the antibodies isolated from step b with a control mammal that has been inoculated with the vaccine of claim 3 wherein the control mammal only has antibodies to FMDV VP1-4 proteins and the infected FMDV mammal has antibodies to FMDV VP1-4 proteins and FMDV nonstructural proteins. The nonstructural proteins may be FMDV 2C, 3A, and 3D polymerase.

Isolated nucleic acid molecules comprising a sequence encoding a protein having one or more sequences selected from the group consisting of: one or more of SEQ ID NOs: 2, 4, 6, 8, 10, 12, 14, 16, 18, 20, 22, 24, 26, 28, 30, 32, 34, 36, 38, 40 and 42 with or without a leader sequence, complements thereof, immunogenic fragments thereof comprising at least 20 amino acids, variants with 80% or more homology to SEQ ID NOs: 2, 4, 6, 8, 10, 12, 14, 16, 18, 20, 22, 24, 26, 28, 30, 32, 34, 36, 38, 40 and 42, complements thereof, immunogenic fragments thereof comprising at least 20 amino acids, and complements thereof are provided

In some embodiments, the nucleic acid sequences is selected from the group consisting of: SEQ ID NOS: 1, 3, 5, 7, 9, 11, 13, 15, 17, 19, 21, 23, 25, 27, 29, 31, 33, 35, 37, 39 and 41 with or without coding sequence for a leader sequence, complements thereof, fragments thereof encoding at least 20 amino acids, complements thereof, nucleic acid molecules 80% homologous to SEQ ID NOS: 1, 3, 5, 7, 9, 11, 13, 15, 17, 19, 21, 23, 25, 27, 29, 31, 33, 35, 37, 39 and 41, complements thereof, fragments thereof encoding at least 20 amino acids, and complements thereof

Vaccine comprising such nucleic acid molecules and/or one or more proteins selected from the group consisting of SEQ ID NOs: 2, 4, 6, 8, 10, 12, 14, 16, 18, 20, 22, 24, 26, 28, 30, 32, 34, 36, 38, 40 and 42 with or without a leader sequence, immunogenic fragments thereof comprising at least 20 amino acids, variants with 80% or more homology to SEQ ID NOs: 2, 4, 6, 8, 10, 12, 14, 16, 18, 20, 22, 24, 26, 28, 30, 32, 34, 36, 38, 40 and 42, and immunogenic fragments thereof comprising at least 20 amino acids are provided.

Also provided are compositions comprising one or more proteins selected from the group consisting of SEQ ID NOs: 2, 4, 6, 8, 10, 12, 14, 16, 18, 20, 22, 24, 26, 28, 30, 32, 34, 36, 38, 40 and 42 with or without a leader sequence, immunogenic fragments thereof comprising at least 20 amino acids, variants with 80% or more homology to SEQ ID NOs: 2, 4, 6, 8, 10, 12, 14, 16, 18, 20, 22, 24, 26, 28, 30, 32, 34, 36, 38, 40 and 42, and immunogenic fragments thereof comprising at least 20 amino acids

Methods of eliciting an immune response against one or more FMDV virus subtypes in a mammal are provided. The methods comprising using a vaccine disclosed here and, in some embodiments, may include the steps of administering a nucleic acid molecule encoding a protein having FMDV immunogenic sequence to the tissue of the mammal; and electroporating cells of the tissue with a pulse of energy at a constant current effective to permit entry of the DNA plasmid into the cells.

A method of diagnosing a mammals infected with FMDV in mammal vaccinated according to processes disclosed herein are also provided. The methods comprise isolating a fluid sample from the vaccinated mammal and detecting the presence of FMDV proteins not included in said vaccine and/or antibodies against FMDV proteins not included in said vaccine. The presence of such FMDV proteins and/or antibodies against such FMDV proteins indicates the vaccinated mammal has been infected with FMDV.

### DETAILED DESCRIPTION

Consensus amino acid sequences have been generated for fusion proteins comprising multiple FMDV proteins and individual FMDV proteins from various scrotypes. Nucleic acid molecules encoding the proteins have also been generated In one aspect of the present invention, there are fusion proteins comprising consensus FMDV proteins VP1, VP2, VP3, VP4 and/or 3C and nucleic acid sequences encoding these proteins, which can be generated and used in a vaccine to provide protection of mammals against foot-and-mouth disease across one or more subtypes of FMDV, including A, Asia 1, O, C, SAT1, SAT2, and SAT3.

In another aspect of the present invention, there are fusion proteins comprising consensus FMDV proteins VP1 and nucleic acid sequences encoding these proteins, from two different subtypes which can be generated and used in a vaccine to provide protection of mammals against foot-and-mouth disease across one or more subtypes of FMDV, including A, Asia 1, O, C, SAT1, SAT2, and SAT3.

In another aspect of the present invention, there are consensus FMDV proteins VP1 and nucleic acid sequences encoding them which can be generated and used in a vaccine to provide protection of mammals against foot-and-mouth disease across one or more subtypes of FMDV, including A, Asia 1, O, C, SAT1, SAT2, and SAT3.

While not being bound by scientific theory, a vaccine directed against the consensus amino acid sequences of VP1, VP2, VP3, and/or VP4 for one or more subtypes of FMDV will present a large repertoire of epitopes that are effective in eliciting an effective immune response (either humoral, cellular or both) against a majority of the species within each subtype of FMDV. This invention relates to using these consensus amino acid VP1, VP2, VP3, and/or VP4 sequences of the FMDV subtypes to generate suitable plasmids and proteins to be used in vaccines for administering to mammals to provide a preventive protection against FMDV. Also, this invention relates to a diagnostic method using these consensus sequences of FMDV VP1, VP2, VP3, and/or VP4 antigens to identify and distinguish mammals that have been properly vaccinated and are uninfected vs. mammals that have been infected with FMDV via the detection of antibodies directed to nonstructural proteins of FMDV such as the 3D polymerase.

While not being bound by scientific theory, VP1 is an excellent immunogenic target for a vaccine directed against the consensus amino acid sequences of VP1. VP1 is a predominant immunogen.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

a. Adjuvant

“Adjuvant” as used herein may mean any molecule added to the DNA plasmid vaccines described herein to enhance antigenicity of the foot-and-mouth disease virus (FMDV) antigen encoded by the DNA plasmids and encoding nucleic acid sequences described hereinafter.

b. Antibody

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

c. Coding Sequence

“Coding sequence” or “encoding nucleic acid” as used herein may mean refers to the nucleic acid (RNA or DNA molecule) that comprise a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

d. Complement

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

e. Consensus or Consensus Sequence

“Consensus” or “consensus sequence” as used herein may mean a synthetic nucleic acid sequence, or corresponding polypeptide sequence, constructed based on analysis of an alignment of multiple subtypes of a particular influenza antigen, that can be used to induce broad immunity against multiple subtypes or serotypes of a particular influenza antigen. Consensus FMDV antigens may include VP1, VP2, VP3, VP4, and C2 protease nucleotide and amino acid sequences. Also, synthetic antigens such as fusion proteins may be manipulated to consensus sequences (or consensus antigens).

f. Constant Current

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

g. Current Feedback or Feedback

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

h. Decentralized Current

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

i. Electroporation

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

j. Feedback Mechanism

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

k. Fragment

“Fragment” as used herein may mean a portion or a nucleic acid that encodes a polypeptide capable of eliciting an immune response in a mammal substantially similar to that of the non-fragment for at least one FMDV subtype such as A, Asia 1, C, O, SAT1, SAT2, or SAT3. The fragments may be DNA fragments selected from at least one of the various encoding nucleotide sequences of the present invention, including SEQ ID NOS: 1-7, and 15-21. The fragments may comprise at least 10%, at least 20%, at least 30%, at least 40%, at least 50%, at least 60%, at least 70%, at least 80%, at least 90% or at least 95% of the nucleic acid sequence of SEQ ID NOS: 1, 3, 5, 7, 9, 11, 13, 15, 17, 19, 21, 23, 25, 27, 29, 31, 33, 35, 37, 39 and 41. Fragments of may comprise at least 10%, at least 20%, at least 30%, at least 40%, at least 50%, at least 60%, at least 70%, at least 80%, at least 90% or at least 95% of SEQ ID NOS: 1, 3, 5, 7, 9, 11, 13, 15, 17, 19, 21, 23, 25, 27, 29, 31, 33, 35, 37, 39 and 41 provided the fragments include one or more of amino acids 21, 86, 127, 129, 154, 156, 182, 195, 206, 218, 220, 237, 249, 255, 265, 271 or 275. All such fragments may also optionally exclude amino acids The DNA fragments may be 30 or more nucleotides in length, 45 or more, 60 or more, 75 or more, 90 or more, 120 or more, 150 or more, 180 or more, 210 or more, 240 or more, 270 or more, 300 or more, 360 or more, 420 or more, 480 or more, 540 or more, 600 or more, 660 or more, 720 or more, 780 or more, 840 or more, 900 or more, 960 or more, 1020 or more, 1080 or more, 1140 or more, 1200 or more, 1260 or more, 1320 or more, 1380 or more, 1440 or more, 1500 or more, 1560 or more, 1620 or more, 1680 or more, 1740 or more, 1800 or more, 1860 or more, 1820 or more, 1880 or more, 1940 or more, 2000 or more, 2600 or more, 2700 or more, 2800 or more, 2900 or more, 2910 or more, 2920 or more, 2930 or more, 2931 or more, 2932 or more, 2933 or more, 2934 or more, 2935 or more, 2936 or more, 2937 or more, or 2938 or more in length

DNA fragments may comprise coding sequences for the immunoglobulin leader such as IgE or IgG sequences.

DNA fragments may be fewer than 10 nucleotides, fewer than 20, fewer than 30, fewer than 40, fewer than 50, fewer than 60, fewer than 75, fewer than 90, fewer than 120, fewer than 150, fewer than 180, fewer than 210, fewer than 240, fewer than 270, fewer than 300, fewer than 360, fewer than 420, fewer than 480, fewer than 540, fewer than 600, fewer than 660, fewer than 720, fewer than 780, fewer than 840, fewer than 900, fewer than 960, fewer than 1020, fewer than 1080, fewer than 1140, fewer than 1200, fewer than 1260, fewer than 1320, fewer than 1380, fewer than 1440, fewer than 1500, fewer than 1560, fewer than 1620, fewer than 1680, or fewer than 1740 nucleotides, fewer than 1800, fewer than 1860, fewer than 1820, fewer than 1880, fewer than 1940, fewer than 2000, fewer than 2600, fewer than 2700, fewer than 2800, fewer than 2900, fewer than 2910, fewer than 2920, fewer than 2930, fewer than 2931, fewer than 2932, fewer than 2933, fewer than 2934, fewer than 2935, fewer than 2936, fewer than 2937, or fewer than 2938.

“Fragment” may also mean a polypeptide fragment that is capable of eliciting an immune response in a mammal substantially similar to that of the non-fragment for at least one FMDV subtype such as A, Asia 1, C, O, SAT1, SAT2, or SAT3. The fragment may be polypeptide fragment selected from at least one of the various encoding polypeptide sequences of the present invention, including SEQ ID NOs: 2, 4, 6, 8, 10, 12, 14, 16, 18, 20, 22, 24, 26, 28, 30, 32, 34, 36, 38, 40 and 42. Polypeptide fragment may be analyzed to contact at least one antigenic epitope as provided by a publicly available database such as the Los Alamos National Laboratory's FMDV Sequence Database. Fragments of proteins may comprise at least 10%, at least 20%, at least 30%, at least 40%, at least 50%, at least 60%, at least 70%, at least 80%, at least 90% or at least 95% of SEQ ID NOs: 2, 4, 6, 8, 10, 12, 14, 16, 18, 20, 22, 24, 26, 28, 30, 32, 34, 36, 38, 40 and 42. Polypeptides may comprise amino acid sequences for the immunoglobulin leader such as IgE or IgG. The polypeptide fragments may be 30 or more amino acids in length, 45 or more, 60 or more, 75 or more, 90 or more, 120 or more, 150 or more, 180 or more, 210 or more, 240 or more, 270 or more, 300 or more, 360 or more, 420 or more, 480 or more, 540 or more, 600 or more, 660 or more, or 710 amino acids or more in length Polypeptide fragments may be fewer than 10 amino acids, fewer than 20, fewer than 30, fewer than 40, fewer than 50, fewer than 60, fewer than 75, fewer than 90, fewer than 120, fewer than 150, fewer than 180, fewer than 210, fewer than 240, fewer than 270, fewer than 300, fewer than 360, fewer than 420, fewer than 480, fewer than 540, fewer than 600, fewer than 660, fewer than 700, fewer than 701, fewer than 702, fewer than 703, fewer than 704, fewer than 705, fewer than 706, fewer than 707, fewer than 708, fewer than 709, or fewer than 710 amino acids in length.
l. Homology

Homology of multiple sequence alignments may generated using ClustalW (httn://www.ebi.ac.uk/Tools/clustalw2/index.html).

m. Identical

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

n. Impedance

“Impedance” as used herein may be used when discussing the feedback mechanism and can be converted to a current value according to Ohm's law, thus enabling comparisons with the preset current.

o. Immune Response

“Immune response” as used herein may mean the activation of a host's immune system, e.g., that of a mammal, in response to the introduction of FMDV consensus antigen via the provided DNA plasmid vaccines. The immune response can be in the form of a cellular or humoral response, or both.

p. Nucleic Acid

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or 1. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

q. Operably Linked

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

r. Promoter

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

s. Stringent Hybridization Conditions

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

t. Substantially Complementary

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

u. Substantially Identical

“Substantially identical” as used herein may mean that a first and second sequence are at least 60%, 65%, 70%, 75%, 80%, 85%, 90%, 95%, 97%, 98% or 99% identical over a region of 8,9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

v. Subtype or Serotype

“Subtype” or “serotype” as used herein interchangeably and in reference to FMDV viruses, and means genetic variants of a FMDV virus antigen such that one subtype is recognized by an immune system apart from a different subtype.

w. Variant

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

x. Vector

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

### 2. FMDV Proteins

Provided herein is an antigen capable of eliciting an immune response in a mammal against one or more foot-and-mouth disease virus (FMDV) subtypes. The antigen may be a FMDV antigen comprising capsid protein VP1, VP2, VP3, VP4, a consensus thereof, a variant thereof, a fragment thereof or a combination thereof. The FMDV antigen may be from FMDV subtype A, Asia 1, C, O, SAT1, SAT2, or SAT3. The FMDV antigen may contain at least one antigenic epitope that may be effective against particular FMDV immunogens against which an immune response can be induced. The empty viral capsid proteins VP1-4 of the FMDV antigen provides an entire repertoire of immunogenic sites and epitopes present in an intact FMDV virus. The consensus FMDV antigen sequence may be derived from FMDV antigen sequences from a plurality of FMDV viruses of one FMDV subtype. The consensus FMDV antigen may comprise VP1, VP2, VP3, and VP4 FMDV subtype consensus protein sequences, which may be a consensus VP1-4 protein. The consensus VP1-4 protein may comprise at least one FMDV protein 3C cleavage site. The protein 3C cleavage site may be present in between each of consensus VP1, VP2, VP3, and VP4 sequences of the consensus VP1-4 protein. Cleavage of the consensus VP1-4 protein by protein 3C may cleave the consensus VP1-4 protein to produce a consensus VP1-, a consensus VP2-, a consensus VP3-, and a consensus VP4 protein. Alternatively, a native proteolytic cleavage site can be present in between each of the consensus antigen sequences, such as the amino acid sequence: SEQ ID NO:45: RGRKRRS.

Fusion proteins comprising consensus VP1, VP2, VP3 and VP4, and a consensus of protease 3C are provided. The are SEQ ID NOs: 2, 4, 6, 8, 10, 12 and 14 which are consensus sequences of subtypes A, Asia 1, C, O, SAT1, SAT2 and SAT3 respectively.

SEQ ID NO:16 is a consensus 3C protease sequence.

Fusion proteins comprising consensus VP1, VP2, VP3 and VP4 are provided. The are SEQ ID NOs: 18, 20, 22, 24, 26, 28 and 30, which are consensus sequences of subtypes A, Asia 1, C, O, SAT1, SAT2 and SAT3 respectively.

SEQ ID NOs:32, 34, 36, and 38 are consensus sequences for VP1 subtypes Asia, O, A and C, respectively. These sequences include the IgE leader sequence SEQ ID NO:44 which may in each case be substituted with a different leader or deleted and substituted with methionine.

SEQ ID NOs:40 and 42 are fusion proteins of two consensus sequences for VP1. SEQ ID NO:40 is consensus VP1 subtypes A and VP1 subtype C. SEQ ID NO:42 is consensus VP1 subtypes Asia and VP1 subtype O. These sequences include the IgE leader sequence SEQ ID NO:44 which may in each case be substituted with a different leader or deleted and substituted with methionine.

Additionally, proteins may be fragments of SEQ ID NOs: 2, 4, 6, 8, 10, 12, 14, 16, 18, 20, 22, 24, 26, 28, 30, 32, 34, 36, 38, 40 and 42. In some embodiments, proteins are 20% of the consensus protein. In some embodiments, proteins are 20% of the consensus protein. In some embodiments, proteins are 30% of the consensus protein. In some embodiments, proteins are 40% of the consensus protein. In some embodiments, proteins are 50% of the consensus protein. In some embodiments, proteins are 60% of the consensus protein. In some embodiments, proteins are 70% of the consensus protein. In some embodiments, proteins are 80% of the consensus protein. In some embodiments, proteins are 90% of the consensus protein. In some embodiments, proteins are 95% of the consensus protein. In some embodiments, proteins are 96% of the consensus protein. In some embodiments, proteins are 97% of the consensus protein. In some embodiments, proteins are 98% of the consensus protein. In some embodiments, proteins are 99% of the consensus protein.

Additionally, proteins may be homologous to SEQ ID NOs: 2, 4, 6, 8, 10, 12, 14, 16, 18, 20, 22, 24, 26, 28, 30, 32, 34, 36, 38, 40 and 42. In some embodiments, proteins are 80% homologous. In some embodiments, proteins are 90% homologous. In some embodiments, proteins are 95% homologous. In some embodiments, proteins are 96% homologous. In some embodiments, proteins are 97% homologous. In some embodiments, proteins are 98% homologous. In some embodiments, proteins are 99% homologous.

Additionally, proteins may be fragments of proteins homologous to SEQ ID NOs: 2, 4, 6, 8, 10, 12, 14, 16, 18, 20, 22, 24, 26, 28, 30, 32, 34, 36, 38, 40 and 42. In some embodiments, proteins are 20% of the homologous protein. In some embodiments, proteins are 20% of the homologous protein. In some embodiments, proteins are 30% of the homologous protein. In some embodiments, proteins are 40% of the homologous protein. In some embodiments, proteins are 50% of the homologous protein. In some embodiments, proteins are 60% of the homologous protein. In some embodiments, proteins are 70% of the homologous protein. In some embodiments, proteins are 80% of the homologous protein. In some embodiments, proteins are 90% of the homologous protein. In some embodiments, proteins are 95% of the homologous protein. In some embodiments, proteins are 96% of the homologous protein. In some embodiments, proteins are 97% of the homologous protein. In some embodiments, proteins are 98% of the homologous protein. In some embodiments, proteins are 99% of the homologous protein.

### 3. Coding Sequences

Provided herein are coding sequences of antigens capable of eliciting an immune response in a mammal against one or more foot-and-mouth disease virus (FMDV) subtypes. The antigen may be a FMDV antigen comprising capsid protein VP1, VP2, VP3, VP4, a consensus thereof, a variant thereof, a fragment thereof or a combination thereof. The FMDV antigen may be from FMDV subtype A, Asia 1, C, O, SAT1, SAT2, or SAT3. The FMDV antigen may contain at least one antigenic epitope that may be effective against particular FMDV immunogens against which an immune response can be induced. The empty viral capsid proteins VP1-4 of the FMDV antigen provides an entire repertoire of immunogenic sites and epitopes present in an intact FMDV virus. The consensus FMDV antigen sequence may be derived from FMDV antigen sequences from a plurality of FMDV viruses of one FMDV subtype. The consensus FMDV antigen may comprise VP1, VP2, VP3, and VP4 FMDV subtype consensus protein sequences, which may be a consensus VP1-4 protein. The consensus VP1-4 protein may comprise at least one FMDV protein 3C cleavage site. The protein 3C cleavage site may be present in between each of consensus VP1, VP2, VP3, and VP4 sequences of the consensus VP1-4 protein. Cleavage of the consensus VP1-4 protein by protein 3C may cleave the consensus VP1-4 protein to produce a consensus VP1-, a consensus VP2-, a consensus VP3-, and a consensus VP4 protein. Alternatively, a native proteolytic cleavage site can be present in between each of the consensus antigen sequences, such as the amino acid sequence: SEQ ID NO:45: RGRKRRS.

Coding sequences for fusion proteins comprising consensus VP1, VP2, VP3 and VP4, and a consensus of protease 3C are provided. The are SEQ ID NOs: 1, 3, 5, 7, 9, 11 and 13 which encode consensus sequences of subtypes A, Asia 1, C, O, SA T1, SAT2 and SAT3 respectively.

SEQ ID NO:15 encodes a consensus 3C protease sequence.

Coding sequences for fusion proteins comprising consensus VP1, VP2, VP3 and VP4 are provided. The are SEQ ID NOs: 17, 19, 21, 23, 25, 27 and 29, which are consensus sequences of subtypes A, Asia 1, C, O, SAT1, SAT2 and SAT3 respectively.

SEQ ID NOs:31, 33, 35, and 37 encode consensus sequences for VP1 subtypes Asia, O, A and C, respectively. These sequences include coding sequences for the IgE leader sequence SEQ ID NO:44 which may in each case be substituted with coding sequence for a different leader or deleted and substituted an initiation codon only.

SEQ ID NOs:40 and 42 are fusion proteins of two consensus sequences for VP1. SEQ ID NO:40 is consensus VP1 subtypes A and VP1 subtype C. SEQ ID NO:42 is consensus VP1 subtypes Asia and VP1 subtype O. These sequences include the IgE leader sequence SEQ ID NO:44 which may in each case can be substituted with coding sequence for a different leader or deleted and substituted an initiation codon only.

Additionally, coding sequences may encode proteins may be fragments of SEQ ID NOs: 2, 4, 6, 8, 10, 12, 14, 16, 18, 20, 22, 24, 26, 28, 30, 32, 34, 36, 38, 40 and 42. In some embodiments, coding sequences encode proteins that are 20% of the consensus protein. In some embodiments, coding sequences encode proteins that are 30% of the consensus protein. In some embodiments, coding sequences encode proteins that are 40% of the consensus protein. In some embodiments, coding sequences encode proteins that are 50% of the consensus protein. In some embodiments, coding sequences encode proteins that are 60% of the consensus protein. In some embodiments, coding sequences encode proteins that are 70% of the consensus protein. In some embodiments, coding sequences encode proteins that are 850% of the consensus protein. In some embodiments, coding sequences encode proteins that are 90% of the consensus protein. In some embodiments, coding sequences encode proteins that are 95% of the consensus protein. In some embodiments, coding sequences encode proteins that are 96% of the consensus protein. In some embodiments, coding sequences encode proteins that are 97% of the consensus protein. I

Additionally, coding sequences may encode proteins that are homologous to SEQ ID NOs: 2, 4, 6, 8, 10, 12, 14, 16, 18, 20, 22, 24, 26, 28, 30, 32, 34, 36, 38, 40 and 42. In some embodiments, coding sequences encode proteins that are 80% homologous. In some embodiments, coding sequences encode proteins that are 90% homologous. In some embodiments, coding sequences encode proteins that are 95% homologous. In some embodiments, coding sequences encode proteins that are 96% homologous. In some embodiments, coding sequences encode proteins that are 97% homologous. In some embodiments, coding sequences encode proteins that are 98% homologous. In some embodiments, coding sequences encode proteins that are 99% homologous.

Additionally, coding sequences encode proteins that are fragments of proteins homologous to SEQ ID NOs: 2, 4, 6, 8, 10, 12, 14, 16, 18, 20, 22, 24, 26, 28, 30, 32, 34, 36, 38, 40 and 42. In some embodiments, coding sequences encode proteins that are 20% of the homologous protein. In some embodiments, coding sequences encode proteins that are 30% of the homologous protein. In some embodiments, coding sequences encode proteins that are 40% of the homologous protein. In some embodiments, coding sequences encode proteins that are 50% of the homologous protein. In some embodiments, coding sequences encode proteins that are 60% of the homologous protein. In some embodiments, coding sequences encode proteins that are 70% of the homologous protein. In some embodiments, coding sequences encode proteins that are 80% of the homologous protein. In some embodiments, coding sequences encode proteins that are 90% of the homologous protein. In some embodiments, coding sequences encode proteins that are 95% of the homologous protein. In some embodiments, coding sequences encode proteins that are 96% of the homologous protein. In some embodiments, coding sequences encode proteins that are 97% of the homologous protein. In some embodiments, coding sequences encode proteins that are 98% of the homologous protein. In some embodiments, coding sequences encode proteins that are 99% of the homologous protein.

Additionally, coding sequences may be fragments of SEQ ID NOs: 1, 3, 5, 7, 9, 11, 13, 15, 17, 19, 21, 23, 25, 27, 29, 31, 33, 35, 37, 39 and 41. In some embodiments, fragments are 20%, 30%, 40%, 50%0, 60% 70%0, 80% 90%, 95%, 96%, 97%, 98% or 99% of SEQ ID NOs: 1, 3, 5,7,9, 11, 13, 15, 17, 19,21,23,25,27,29,31,33,35,37,39 and 41.

Additionally, coding sequences may be homologous to SEQ ID NOs: 1, 3, 5, 7, 9, 11, 13, 15, 17, 19, 21, 23, 25, 27, 29, 31, 33, 35, 37, 39 and 41. In some embodiments, coding sequences are 80%, 90%, 95%, 96%, 97%, 98% or 99% homologous to SEQ ID NOs: 1, 3, 5, 7, 9, 11, 13, 15, 17, 19, 21, 23, 25, 27, 29, 31, 33, 35, 37, 39 and 41.

Additionally, coding sequences may be homologous to fragments of SEQ ID NOs: 1, 3, 5, 7, 9, 11, 13, 15, 17, 19, 21, 23, 25, 27, 29, 31, 33, 35, 37, 39 and 41. In some embodiments, fragments are 20%, 30%, 40%, 50%, 60%, 70%, 80%, 90%, 95%, 96%, 97%, 98% or 99% of SEQ ID NOs: 1, 3, 5, 7, 9, 11, 13, 15, 17, 19, 21, 23, 25, 27, 29, 31, 33, 35, 37, 39 and 41. and the coding sequences are 80%, 90%, 95%, 96%, 97%, 98% or 99% homologous to the fragments of SEQ ID NOs: 1, 3, 5, 7, 9, 11, 13, 15, 17, 19, 21, 23, 25, 27, 29, 31, 33, 35, 37, 39 and 41.

### 4. Plasmid

Provided herein is a vector that is capable of expressing one or more FMDV antigens in the cell of a mammal in a quantity effective to elicit an immune response in the mammal. The vector may comprise heterologous nucleic acid encoding the FMDV antigen. The vector may be a plasmid. The plasmid may be useful for transfecting cells with nucleic acid encoding a FMDV antigen, which the transformed host cell is cultured and maintained under conditions wherein expression of the FMDV antigen takes place.

The plasmid may comprise a nucleic acid encoding a FMDV antigen selected from the group consisting of: SEQ ID NO: 2, 4, 6, 8, 10, 12, 14, 16, 18, 20, 22, 24, 26, 28, 30, 32, 34, 36, 38, 40 and 42, fragments thereof, homologous sequences thereof and fragments of homologous. The plasmid may further comprise an initiation codon or leader sequence, which may be upstream of the coding sequence, and a stop codon, which may be downstream of the coding sequence. The initiation and termination codon may be in frame with the coding sequence.

The plasmid may also comprise a promoter that is operably linked to the coding sequence. The promoter operably linked to the coding sequence a may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may also comprise a regulatory sequence, which may be well suited for gene expression in a cell into which the plasmid is administered. The coding sequence may comprise a codon, which may allow more efficient transcription of the coding sequence in the host cell.

The coding sequence may comprise an Ig leader sequence. The leader sequence may be 5′ of the coding sequence. The consensus protein encoded by this sequence may comprise an N-terminal Ig leader followed by a consensus protein. The N-terminal Ig leader may be IgE or IgG.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNA I or pcDNA3 (Invitrogen, San Diego, Calif.), which maybe used for protein production in mammalian cells such as Chinese hamster ovary (CHO) cells.

Plasmids may comprise one or more coding sequences encoding one or more of VP1, VP2, VP3, VP4, and 3C from one or more subtypes such as Asia, A, O, C, SAT1, SAT2 and SAT3.

In some embodiments, a plasmid comprises coding sequences for multiple distinct consensus FMDV antigens VP1, VP2, VP3, VP4 and 3C from subtype Asia, A, O, C, SAT1, SAT2 or SAT3.

In some embodiments, a plasmid comprises coding sequences for multiple distinct consensus FMDV antigens VP1, VP2, VP3 and VP4 from subtype Asia, A, O, C, SAT1, SAT2 or SAT3.

In some embodiments, a plasmid comprises coding sequences for two distinct consensus FMDV antigen VP1 from two of subtypes Asia, A, O, and C such as VP1 from subtype Asia VP1 from subtype O, or a VP1 from t subtype A and VP1 from subtype C.

In some embodiments, a plasmid comprises coding sequences for a consensus FMDV antigen VP1 such as VP1 subtype Asia, VP1 subtype A, VP1 subtype O or VP1 subtype C.

The coding sequence can be encoded by a distinct DNA plasmid, all regulated by an operably linked promoter, e.g., a DNA plasmid having an encoding sequence regulated by one or mote promoters the encoding sequence comprising multiple consensus FMDV antigens.

### 5. Vaccine

While not being bound by scientific theory, a vaccine that can be used to elicit an immune response (humoral, cellular, or both) broadly against FMDV may comprise one or more coding sequences set forth above, i.e. nucleic acid sequences that encodes one or more proteins VP1, VP2, VP3, CVP4 and 3C from subtypes selected from the group consisting of: FMDV subtypes such as A, Asia 1, C, O, SAT1, SAT2, SAT3, or combinations thereof Coding sequences may also include those that comprise homologous sequences, fragments, and homologous sequences of fragments. Alternatively or in addition, compositions which induce anti-FMDV immune response may comprise one or more proteins selected from the group consisting of: FMDV subtypes such as A, Asia 1, C, O, SAT1, SAT2, SAT3, or combinations thereof.

Provided herein is a vaccine capable of generating in a mammal an immune response against one or more FMDV subtypes. The vaccine may comprise the plasmid as discussed above. The vaccine may comprise a plurality of the plasmids each directed to one or more FMDV subtypes such as A, Asia 1, C, O, SAT1, SAT2, SAT3, or combinations thereof. The vaccine may also comprise the FMDV antigens themselves directed against one or more FMDV subtypes such as A, Asia 1, C, O, SAT1, SAT2, SAT3, or combinations thereof. The vaccine may also comprise plasmids directed to FMDV subtypes from particular regions in the world, for example, Asia, Europe and sub-Africa. Alternatively or in addition, the vaccine may comprise proteins of one or more FMDV subtypes such as A, Asia 1, C, O, SAT1, SAT2, SAT3, or combinations thereof. The vaccine may also comprise the FMDV antigens themselves directed against one or more FMDV subtypes such as A, Asia 1, C, O, SAT1, SAT2, SAT3, or combinations thereof. The vaccine may also comprise plasmids and/or proteins directed to FMDV subtypes from particular regions in the world, for example, Asia, Europe and sub-Africa. The vaccine may be provided to induce a therapeutic or prophylactic immune response.

The vaccine may also comprise a nucleic acid encoding a FMDV C3 protease, which may be a consensus C3 protease nucleic acid. The consensus protein 3C nucleic acid may be a protein 3C coding sequence. Alternatively or in addition, the vaccine may also comprise FMDV C3 protease, such as a consensus C3 protease for example a protein 3C. The vaccine may also comprise a chimeric gene encoding full or partial VP1-4 coding sequence and full or partial C3 coding sequence. Alternatively or in addition, the vaccine may also comprise a fusion protein comprising full or partial VP1-4 and full or partial C3.

Provided herein are pharmaceutical compositions according to the present invention which comprise about 1 nanogram to about 10 mg of DNA. In some embodiments, pharmaceutical compositions according to the present invention comprise from between: 1) at least 10, 15, 20, 25, 30, 35, 40, 45, 50, 55, 60, 65, 70, 75, 80, 85, 90, 95 or 100 nanograms, or at least 1,5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms, or at least 1.5, 2, 2.5, 3, 3.5, 4, 4.5, 5, 5.5, 6, 6.5, 7, 7.5, 8, 8.5, 9, 9.5 or 10 mg or more; and 2) up to and including 15, 20, 25, 30, 35, 40, 45, 50, 55, 60, 65, 70, 75, 80, 85, 90, 95 or 100 nanograms, or up to and including 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms, or up to and including 1.5, 2, 2.5, 3, 3.5, 4, 4.5, 5, 5.5, 6, 6.5, 7, 7.5, 8, 8.5, 9, 9.5 or 10 mg. In some embodiments, pharmaceutical compositions according to the present invention comprise about 5 nanogram to about 10 mg of DNA. In some embodiments, pharmaceutical compositions according to the present invention comprise about 25 nanogram to about 5 mg of DNA. In some embodiments, the pharmaceutical compositions contain about 50 nanograms to about 1 mg of DNA. In some embodiments, the pharmaceutical compositions contain about 0.1 to about 500 micrograms of DNA. In some embodiments, the pharmaceutical compositions contain about 1 to about 350 micrograms of DNA. In some embodiments, the pharmaceutical compositions contain about 5 to about 250 micrograms of DNA. In some embodiments, the pharmaceutical compositions contain about 10 to about 200 micrograms of DNA. In some embodiments, the pharmaceutical compositions contain about 15 to about 150 micrograms of DNA. In some embodiments, the pharmaceutical compositions contain about 20 to about 100 micrograms of DNA. In some embodiments, the pharmaceutical compositions contain about 25 to about 75 micrograms of DNA. In some embodiments, the pharmaceutical compositions contain about 30 to about 50 micrograms of DNA. In some embodiments, the pharmaceutical compositions contain about 35 to about 40 micrograms of DNA. In some embodiments, the pharmaceutical compositions contain about 100 to about 200 microgram DNA. In some embodiments, the pharmaceutical compositions comprise about 10 microgram to about 100 micrograms of DNA. In some embodiments, the pharmaceutical compositions comprise about 20 micrograms to about 80 micrograms of DNA. In some embodiments, the pharmaceutical compositions comprise about 25 micrograms to about 60 micrograms of DNA. In some embodiments, the pharmaceutical compositions comprise about 30 nanograms to about 50 micrograms of DNA. In some embodiments, the pharmaceutical compositions comprise about 35 nanograms to about 45 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

Preferably the pharmaceutical composition is a vaccine, and more preferably a DNA vaccine.

The vaccine may be a DNA vaccine. The DNA vaccine may comprise a plurality of the same or different plasmids comprising nucleic acid coding sequences for one or more of consensus prostate antigens. The DNA vaccine may comprise one or more nucleic acid sequences that encode one or more of consensus prostate antigens. When the DNA vaccine comprises coding sequences of more than one consensus prostate antigens all such sequences may be present on a single plasmid, or each such sequences may be present on a different plasmids.

In some embodiments, vaccines may comprise nucleic acid sequences that encode one or more of consensus prostate antigens in combination with one or more of consensus prostate antigens.

DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. The vaccine can be an RNA of the prostate antigen. The RNA vaccine can be introduced into the cell.

The vaccine can be a recombinant vaccine comprising the genetic construct or antigen described above. The vaccine can also comprise one or more consensus prostate antigens in the form of one or more protein subunits, or one or more attenuated viral particles comprising one or more consensus antigens. The attenuated vaccine can be attenuated live vaccines, killed vaccines and vaccines that use recombinant vectors to deliver foreign genes that encode one or more consensus prostate antigens, and well as subunit and protein vaccines. Examples of attenuated live vaccines, those using recombinant vectors to deliver prostate antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,3 64; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference. Vaccines may comprise plasmids in combination with other vaccine components such as FMDV proteins or expression vectors encoding proteins.

The vaccine provided may be used to induce immune responses including therapeutic or prophylactic immune responses. Antibodies and/or killer T cells may be generated which are directed to the consensus prostate antigen. Such antibodies and cells may be isolated.

The vaccine may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be an adjuvant. The adjuvant may be other genes that are expressed in alternative plasmid or are delivered as proteins in combination with the plasmid above in the vaccine. The adjuvant may be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosac-associated epithelial chemokine (MEC), IL-12, IL-15, MHC, CD80, CD86 including IL-15 having the signal sequence deleted and optionally including the signal peptide from IgE. The adjuvant may be IL-12, IL-15, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof Other genes which may be useful adjuvants include those encoding: MCP-1, MIP-la, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Serial No. 021,579 filed Apr. 1, 1994, which is fully incorporated by reference.

The vaccine may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

### 6. Methods of Delivery the Vaccine

Provided herein is a method for delivering the vaccine for providing genetic constructs and proteins of the FMDV antigen which comprise epitopes that make them particular effective against immunogens of FMDV against which an immune response can be induced. The method of delivering the vaccine or vaccination may be provided to induce a therapeutic and prophylactic immune response. The vaccination process may generate in the mammal an immune response against a plurality of FMDV subtypes. The vaccine may be delivered to an individual to modulate the activity of the mammal's immune system and enhance the immune response. The delivery of the vaccine may be the transfection of the FMDV antigen as a nucleic acid molecule that is expressed in the cell and delivered to the surface of the cell upon which the immune system recognized and induces a cellular, humoral, or cellular and humoral response. The delivery of the vaccine may be use to induce or elicit and immune response in mammals against a plurality of FMDV viruses by administering to the mammals the vaccine as discussed above.

Upon delivery of the vaccine and plasmid into the cells of the mammal, the transfected cells will express and secrete consensus capsids for each of the plasmids injected from the vaccine. These secreted capsid proteins will be recognized as foreign by the immune system and antibodies will be made against them. These antibodies will be maintained by the immune system and allow for rapid clearing of subsequent FMDV challenge.

The vaccine may be administered to a mammal to elicit an immune response in a mammal. The mammal may be human, primate, non-human primate, cow, cattle, sheep, goat, antelope, bison, water buffalo, bison, bovids, deer, hedgehogs, elephants, llama, alpaca, mice, rats, and chicken.

a. Combination Treatments

The vaccine may be administered in combination with other proteins or genes encoding α-interferon, γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE, IL-12, IL-15, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, MCP-1, MIP-la, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p³8, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or combinations thereof. The vaccine may also be administered in combination with CTACK protein, TECK protein, MEC protein or functional fragments thereof.

The vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns”, or other physical methods such as electroporation (“EP”), “hydrodynamic method”, or ultrasound.

The plasmid of the vaccine may be delivered to the mammal by several well known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The FMDV antigen may be delivered via DNA injection and along with in vivo electroporation.

b. Electroporation

Administration of the vaccine via electroporation of the plasmids of the vaccine may be accomplished using electroporation devices that can be configured to deliver to a desired tissue of a mammal a pulse of energy producing a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electronporation may be accomplished using the VGXP Cellectra™ system to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

c. Method of Preparing Vaccine

Provided herein are methods for preparing the vaccine. In some embodiments, the methods are methods of preparing the vaccines comprising DNA plasmids. The DNA plasmids, after the final subcloning step into the mammalian expression plasmid, can be used to inoculate a cell culture in a large scale fermentation tank, using known methods in the art. The plasmid is transformed into a compatible host cell and cultured and maintained under conditions wherein expression of the FMDV antigen takes place. The FMDV antigen may be recovered from the culture either by lysing cells or from the culture medium and isolated. The isolated VP1-4 consensus proteins may be used in the vaccine as a natural source of antibodies. The FMDV antigen may be produce by recombinant techniques using automated synthesizers may also be employed to produce isolated essential pure FMDV antigen. These techniques may be useful for introducing variants of the FMDV antigen for particular subtypes of FMDV.

The DNA plasmids for use with the EP devices of the present invention can be formulated or manufactured using a combination of known devices and techniques, but preferably they are manufactured using an optimized plasmid manufacturing technique that is described in a licensed, co-pending U.S. provisional application U.S. Ser. No. 60/939,792, which was filed on May 23, 2007. In some examples, the DNA plasmids used in these studies can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

d. Method for Preparing VP1-4 Expression Constructs

A multi-targeting FMDV DNA vaccine is constructed by first optimizing VP1, VP2, VP3, and VP4 amino acid sequences for one the FMDV subtypes Asia, O, A, C, SAT1, SAT2, and SAT3 using at least 10 different sequences from the subtype. Nucleic acids each encoding the subtype-optimized VP1-4 proteins are produced. The subtype-optimized VP1-4 nucleic acid sequences are cloned as a contiguous coding sequence, with the VPs separated by intervening FMDV protein 3C protease cleavage sites. The optimized VP1-4 coding sequence is inserted into an expression vector, either pVAX or pAV0242, under the control of an operator. An IgE leader sequence is placed upstream of the optimized VP1-4 coding sequence so that the encoded protein includes an N-terminal IgE leader. Two stop codons are placed at the 3′ end of the VP1-4 coding sequence.

In addition, a nucleic acid encoding the FMDV protein 3C is constructed by optimizing the 3C nucleic acid sequence for one of the FMDV subtypes Asia 1, O, A, C, SAT1, SAT2, and SAT3 using at least 10 different sequences from the subtype. A nucleic acid encoding the subtype-optimized 3C protein is produced, and cloned into a pVAX or pAV0242 plasmid.

e. Method of Using Vaccine as a Marker

Provided herein is also a method of differentiating between a vaccinated mammal with the vaccine and an infected mammal with FMDV. The method may comprise sample from a mammal and isolating the mammals antibodies from the sample. A mammal that has been vaccinated by the vaccine may have antibodies that are specific only for the empty capsid proteins of the FMDV antigen, i.e, viral coat proteins VP1-4 against FMDV subtypes A, Asia I, O, C, SAT1, SAT2, SAT3, or a combination thereof. A mammal that has been infected by FMDV will have antibodies against FMDV viral coat proteins VP1-4 of a particular FMDV subtype such as A, Asia 1, O, C, SAT1, SAT2, or SAT3 and in addition, against antibodies against the non-structural (NS) proteins of FMDV. The NS proteins of FMDV may include the protease 3C protease as well as FMDV protein 2C, 3A, 3B, and 3D (polymerase). The method may comprise identifying an antibody against a NS protein of FMDV such as the highly antigenic 3D protein. The method further comprises comparing to the sera sample of the vaccinated mammals to determine the presence or non-presence of FMDV NS proteins. The infected mammal has antibodies against the NS proteins of FMDV, while the vaccinated mammal does not have antibodies against the NS proteins as this mammal have a sufficient immunity against FMDV infection. The method may comprise differentiating mammals have antibodies to VP1-4 vs. mammals have antibodies to VP1-4 and the 3D polymerase of FMDV.

Generally, an agent may be used. The agent may be VP1-4 or a NS protein such as 3D polymerase. A sample from the mammal is isolated with FMDV antibodies and are reacted against the agent to identify the specificity of the FDMV antibody.

The sample of the method can be isolated from the mammal and may include a serum sample from blood, saliva, tears, cerebrospinal fluid, aqueous humor, pleural fluid, pericardial fluid, lymph node fluid, chime, chyle, bile, urine, synovial fluid, vomit, peritoneal fluid, stool water, semen, amiotic fluid, milk, serum, interstitial fluid, and pancreatic juice.

Methods for performing the diagnostic test include performing a immunprecipitation with [35S] methionine-labeled cell lysates from the mammal, western blots, and immunoblots to particular FMDV proteins such as VP1-4 and 3D polymerase.

The method of detecting described herein may be implemented in a variety of well-known detection systems to determine the presence of antibodies to FMDV VP1-4 or 3D polymerase in a test or control sample. The detection system may comprise a fluorescent or other means comparison between a signal generated from a detection label that is bound to a particular FMDV protein such as VP1-4 and 3D polymerase and a pre-determined value to determine the presence or absence of antibodies to FMDV VP1-4 or 3D polymerase in the test sample. The pre-determined value may be a ratio of the signal measured from the test sample to the signal measured from the control sample. In general, a test sample generating a signal that is three standard deviations above the mean signal measured from a control sample that contains no FMDV 3D polymerase antibodies that may be considered positive for FMDV 3D polymerase and therefore an infected mammal.

Alternatively, an apparatus such as a densitometer may be employed for measuring a numerical value of the detectable label. The pre-determined value may be determined using a Receive Operator Curve (“ROC”) using the method of Sackett et al., Clinical Epidemiology: A Basic Science for Clinical Medicine, p. 106-107 (Little Brown and Co., 1985). The pre-determined value may be based upon relative light units by a fluorescent imager or other means as describe above. Briefly, the pre-determined value may be determined from a plot of pairs of true positive rates (namely, sensitivity) and false positive rates (namely, 100% specificity) that correspond to each possible value for the diagnostic test result. The pre-determined value on the plot that is the closest to the upper left-hand corner (namely, the value that encloses the largest area) is the most accurate pre-determined value, and a sample generating a signal that is higher than the pre-determined value determined by this method may be considered positive. Alternatively, the pre-determined value may be shifted to the left along the plot, to minimize the false positive rate.

### (a) Immunoblot

The method of detecting may be used in an immunoblot detection system to detect antibodies to FMDV VP1-4 or 3D polymerase in a test or control sample. The immunoblot may use a solid support to immobilize the agent.

The immunoblot may use two separate control samples (namely, a first control and a second control), which may be immobilized on a solid support. The immunoblot may use three separate, discrete control samples (namely, a first control, a second control and a third control). If more than one control sample is present, then the controls may be identical to one another or different from one another. Two of the control samples may be identical (such as, for example, the first control and the second control). If two of the control samples are identical, the concentration of one of the control samples (either the first control or the second control or if three controls are present, the level of the first control or the third control or the second control or third control) may be higher (or greater) than the other control. The control sample may be in a higher concentration than the other control and may be referred to as the “high control”. The control immobilized on the strip, disc or sheet in a lower concentration than the high control may be referred to as the “low control”. The ratio of the concentration of low control to high control may be from about 1:2 to about 1:10, preferably, about 1:5 to about 1:6. For example, the first control may be the low control and the second control may be the high control. Alternatively, the first control may be the high control and the second control may be the low control. By way of another example, a three control detection system may comprise a low control and a high control as well a third control (which can be used, for example, to verify sample addition). The low control and high control may be human plasma (wherein the ratio of low control to high control is from about 1:2 to about 1:10) and the third control may be SDB Chagas or human plasma.

In the flow-through format, an immobilized agent on the solid support may be immersed in a solution containing the test sample. Alternatively, the solid support may be placed in a reaction tray along with a diluent and then the test sample added to the reaction tray. The test sample and agent are allowed to incubate for a sufficient period of time using the same times and techniques described previously herein. Unbound test sample may be removed using the techniques described previously herein. In this format, anti-FDMV antibodies to VP1-4 or a NS structure protein such as 3D polymerase within the test sample may bind to the immobilized agent (and the at least one control) as the test sample passes through the membrane. At least one detection reagent (such as a detection reagent described previously herein containing a detectable label) may be added. At least one detection reagent may bind to each of the agent-antibody complexes formed as the solution containing the detection reagent flows through the strip. To determine the presence or absence of anti-FDMV antibodies to VP1-4 or a NS structure protein such as 3D polymerase in the test sample, the detection of the bound detection reagents may be performed as described above using the a cut-off or by comparing the intensity of one or more signals generated by one or more controls as discussed in more detail below.

When a low control and high control as described above may be used in the flow-through format, the presence or absence of the anti-FDMV antibodies to VP1-4 or a NS structure protein such as 3D polymerase in the test sample may be determined by identifying the presence of a signal from the detectable label at each of the test bands (or spots or dots) for the agents. If a signal is identified at a test band for a agent, then the intensity of this detected signal is compared with the intensity of the signal from the low control band (or spot or dot) and the high control band (or spot or dot), using a scale of 0 to 4+. The reading is 0 when no band is visible. The intensities of the low control band and high control band may be defined as 1+(for the low control) and 3+(for the high control), respectively. A test band with an intensity comparable to that of the low control would be rated 1+. A band with intensity between that of the low control and the high control band would be rated 2+. A band with an intensity comparable to that of the high control would be rated 3+. A band intensity higher than that of the high control would be rated 4+.

### (b) Competitive Assay

The method of detecting may be used in a competitive detection system to identify test samples with anti-FDMV antibodies to VP1-4 or a NS structure protein such as 3D polymerase. The agent may be immobilized on a solid support as described above. The immobilized agent may then be contacted with a competitive antibody that is detectable labeled, known to bind the agent, and competes with anti-FDMV antibodies to VP1-4 or a NS structure protein such as 3D polymerase in the test sample. The immobilized agent is also contacted with the test sample. The signal from the detectably labeled antibody may be lower in test samples containing anti-FDMV antibodies to VP1-4 or a NS structure protein such as 3D polymerase because both sets of antibodies are competing for the immobilized agent.

f. Diagnostic Kit

Provided herein is a kit for performing the diagnostic method of identifying mammals that have been vaccinated with the vaccine vs. mammals infected with FMDV. The kit provides materials for allowing one to identify mammals that have been infected with FMDV to identify antibodies against the FS proteins including the 3D polymerase protein of FMDV vs. antibodies only to the empty capsid proteins VP1-4 of a vaccinated mammal. Test kits may include one or more reagents such as the agent useful for practicing one or more immunoassays according to the invention. A test kit generally includes a package with one or more containers holding the reagents, as one or more separate compositions or, optionally, as admixture where the compatibility of the reagents will allow. The test kit may also include other material(s), which may be desirable from a user standpoint, such as a buffer(s), a diluent(s), a standard(s), and/or any other material useful in sample processing, washing, or conducting any other step of the assay.

Kits according to the invention may include a solid phase and an agent affixed to a solid support. Kits may be employed for conducting sandwich immunoassays, and include a labeled detection antibody. The labeled detection antibody may be an anti-human IgG labeled antibody. The kit may further include a detectable label.

The test kit may include at least one direct label, such as acridinium-9-carboxamide. Test kits according to the invention may also include at least one indirect label. If the label employed generally requires an indicator reagent to produce a detectable signal, the test kit may include one or more suitable indicator reagents.

The test kit may include instructions for carrying out one or more of the immunoassays of the invention. Instructions included in kits of the invention may be affixed to packaging material or may be included as a package insert. While the instructions are typically written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### EXAMPLES

### Example 1

### Expression of Recombinant VP1-4

The subtype-optimized VP1-4 protein and optimized 3C protein are expressed by performing an in vitro translation assay using the optimized VP1-4 and 3C expression plasmids above. Translation of these proteins yields the expected bands on a SDS-PAGE gel.

To confirm expression of the VP1-4 proteins, a nucleic acid encoding a subtype-optimized VP1-4 protein and an N-terminal IgE leader is cloned into a HIS-tag bacterial expression vector. A nucleic acid encoding a subtype-optimized 3C protein is also cloned into a HIS-tag bacterial expression vector. The optimized VP1-4 and 3C proteins are expressed using a bacterial expression system, and affinity-purified using Ni-column separation. The purified proteins are analyzed using a SDS-PAGE gel. SDS-PAGE reveals the expected bands.

### Example 2

### Method of Vaccination

To test the efficacy of the DNA plasmids, Balb/C mice are immunized with the optimized VP1-4- and 3C-encoding pVAX plasmids. Empty pVAX and human IL-15-encoding pVAX vectors are used as controls. The mice are immunized three times daily on Days 0, 14, and 28. Immunized mice are sacrificed 3 days after the final immunization. Sera from the mice are collected and analyzed for anti-VP1, -VP2, -VP3, and -VP4 ELISA. The HIS-tagged recombinant proteins from Example 1 are used as the capture antigen. Sera from pVAX control mice fail to recognize any of subtype-optimized VP1-4. In contrast, mice immunized with the subtype-optimized VP1-4 DNA vaccine developed antibodies toward subtype-optimized VP1, -2, -3, and -4, indicating that the optimized VP1-4 fusion vaccine is causing mice to mount an immune response against all four VPs.

### Example 3

### Preparing Expression Constructs

A multi-targeting FMD DNA vaccine was constructed. VP1 sequences from the subtypes Asia1, O, A, C, SAT1, SAT2, and SAT3 were first consensus optimized with at least 10 different sequences from each subtype. Thereafter, two VP1 sequences were inserted under one promoter and were separated by two consecutive cleavage sites.

An IgE leader sequence was inserted in front of the first ORE and two stop codons were inserted after the second ORE. The first plasmid encodes the Asia and O VP1, is 1362 bp.

The second plasmid, which encodes the A and C VP1, is 1356 bp. The third and fourth plasmids target the sub-African subtypes with the first encoding SAT1 and SAT2 VP1 and the second encoding SAT3 VP1.

### Example 4

### Expression of Recombinant VP1-4

The cloned plasmids were then expressed with an in vitro translation assay. Translation of all of the single VP1 constructs—A, Asia, C, and O—yielded the expected bands, [about 24.5 kDa] and the A+C VP1 and Asia+O VP1 constructs yielded a higher dimeric band. They constructs have FLAG-epitopes which were used in immunoprecipitation.

### Example 5

### Method of Vaccination

In order to confirm immune responses against FMD, we generated recombinant FMD VP1 proteins from all four VP1 subtypes (A, Asia, C, and O) Recombinant Consensus FMDV VP1 sequences (IgE Leader sequence is underlined at the N terminus)

The proteins were cloned into a HIS-tagged bacterial expression vector, and vector was expressed. The proteins were purified via Ni-column separation, and the expressed proteins are indicated with an arrow.

Next to test the efficacy of the DNA plasmids, Balb/C mice were immunized. Mice were immunized with 15 pg of DNA per immunization using CELLECTRA electroporation. There were 7 immunization groups:
1. pVax
2. pVax-FMDV VP1 A+pVAX1-IL-15
3. pVax-FMDV VP1 Asia+pVAX1-IL-15
4. pVax-FMDV VP1 C+pVAX1-IL-15
5. pVax-FMDV VP1 O+pVAX1-IL-15
6. pVax-FMDV VP1 A-C+pVAX1-IL-15
7. pVax-FMDV VP1 Asia-O+pVAX1-IL-15

The mice were immunized 3 times on day 0, 14, and 28, and were sacrificed 3 days after the last immunization. Sera from the animals were collected and analyzed for anti-VP1 ELISA. The recombinant proteins were used as the capture antigen. Scra from pVAX control mice failed to recognize A, Asia, C, and O VP1 proteins. Conversely, mice immunized with the A, Asia, C, and O DNA vaccines developed antibodies toward A, Asia, C, and O VP1 proteins, respectively. More importantly, mice immunized with either the VP1 A-C or API Asia-0 vaccines developed antibodies toward all 4 VP1 subtypes, suggesting that the consensus-VP1 fusion vaccine is generating immune responses against all 4 Asian-European FMD subtypes.

1. An isolated nucleic acid comprising a sequence encoding a protein having one or more sequences selected from the group consisting of: one or more of SEQ ID NOs: 2, 4, 6, 8, 10, 12, 14, 16, 18, 20, 22, 24, 26, 28, 30, 32, 34, 36, 38, 40 and 42 with or without a leader sequence, complements thereof, immunogenic fragments thereof comprising at least 20 amino acids, variants with 80% or more homology to SEQ ID NOs: 2, 4, 6, 8, 10, 12, 14, 16, 18, 20, 22, 24, 26, 28, 30, 32, 34, 36, 38, 40 and 42, complements thereof, immunogenic fragments thereof comprising at least 20 amino acids, and complements thereof,

2. The nucleic acid of claim 1, wherein the sequence is selected from the group consisting of: SEQ ID NOS: 1, 3, 5, 7, 9, 11, 13, 15, 17, 19, 21, 23, 25, 27, 29, 31, 33, 35, 37, 39 and 41 with or without coding sequence for a leader sequence, complements thereof, fragments thereof encoding at least 20 amino acids, complements thereof, nucleic acid molecules 80% homologous to SEQ ID NOS: 1, 3, 5, 7, 9, 11, 13, 15, 17, 19, 21, 23, 25, 27, 29, 31, 33, 35, 37, 39 and 41, complements thereof, fragments thereof encoding at least 20 amino acids, and complements thereof

3. The nucleic acid of claim 1 comprising a sequence encoding a protein selected from the group consisting of: SEQ ID NOs: 2, 4, 6, 8, 10, 12, 14, 16, 18, 20, 22, 24, 26, 28, 30, 32, 34, 36, 38, 40 and 42 with or without a leader sequence.

4. The nucleic acid of claim 1 comprising a sequence selected from the group consisting of: SEQ ID NOS: 1, 3, 5, 7, 9, 11, 13, 15, 17, 19, 21, 23, 25, 27, 29, 31, 33, 35, 37, 39 and 41.

5. The nucleic acid of any of claims 1-4 wherein the leader sequence is an Ige leader sequence.

6. The nucleic acid of any of claims 1-5 wherein said nucleic acid is a plasmid.

7. The nucleic acid of any of claims 1-6 wherein said nucleic acid is a plasmid that is an expression vector.

8. A vaccine comprising a nucleic acid of any of claims 1-7 and/or one or more proteins selected from the group consisting of SEQ ID NOs: 2, 4, 6, 8, 10, 12, 14, 16, 18, 20, 22, 24, 26, 28, 30, 32, 34, 36, 38, 40 and 42 with or without a leader sequence, complements thereof, immunogenic fragments thereof comprising at least 20 amino acids, variants with 80% or more homology to SEQ ID NOs: 2, 4, 6, 8, 10, 12, 14, 16, 18, 20, 22, 24, 26, 28, 30, 32, 34, 36, 38, 40 and 42, complements thereof, immunogenic fragments thereof comprising at least 20 amino acids, and complements thereof.

9. The vaccine of claim 8 further comprising an adjuvant.

10. The vaccine of claim 8 further comprising an adjuvant is selected from the group consisting of IL-12 and/or IL-15 or a nucleic acid sequence encoding IL-12 and/or IL-15.

11. A compositions comprising one or more proteins selected from the group consisting of SEQ ID NOs: 2, 4, 6, 8, 10, 12, 14, 16, 18, 20, 22, 24, 26, 28, 30, 32, 34, 36, 38, 40 and 42 with or without a leader sequence, complements thereof, immunogenic fragments thereof comprising at least 20 amino acids, variants with 80% or more homology to SEQ ID NOs: 2, 4, 6, 8, 10, 12, 14, 16, 18, 20, 22, 24, 26, 28, 30, 32, 34, 36, 38, 40 and 42, complements thereof, immunogenic fragments thereof comprising at least 20 amino acids, and complements thereof

12. A method of eliciting an immune response against one or more FMDV virus subtypes in a mammal, comprising administering a vaccine according to claim 8.

13. The method of claim 12 wherein the vaccine comprises a nucleic acid molecule, the method comprising the steps of
a) administering the nucleic acid molecule to the tissue of the mammal; and
b) electroporating cells of the tissue with a pulse of energy at a constant current effective to permit entry of the DNA plasmid into the cells.

14. The method of claim 13 wherein step a) comprises injecting the DNA plasmid vaccine into intradermic, subcutaneous, or muscle tissue.

15. The method of claim 13 or 14 wherein the current is preset for delivering to the tissue and pulse of energy is at a constant current that equals the preset current.

16. The method of any of claims 13-15 wherein the electroporating step further comprises:
(a) measuring the impedence in the electroporated cells;
(b) adjusting the energy level of the pulse of energy relative to the measured impedance to maintain a constant current in the electroporated cells;
wherein the measuring and adjusting steps occur within a lifetime of the pulse of energy.

17. The method of any of claims 13-16 wherein the electroporation step comprise delivering the pulse of energy to a plurality of electrodes according to a pulse sequence pattern that delivers the pulse of energy in a decentralized pattern.

18. The method of any of claims 12-17, wherein the mammal has not been infected with FMDV and the immune response is a protective immune response.

19. The method of any of claims 12-17, wherein the mammal has been infected with FMDV and the immune response is a therapeutic immune response.

20. A method of diagnosing a mammals infected with FMDV in mammal vaccinated with a vaccine of claim 8, the method comprising:
a) isolating a fluid sample from the mammal; and
b) detecting the presence of FMDV proteins not included in said vaccine and/or antibodies against FMDV proteins not included in said vaccine, wherein the presence of FMDV proteins not included in said vaccine and/or antibodies against FMDV proteins not included in said vaccine indicates the mammal has been infected with FMDV.


SEQUENCE LISTING (49 sequences)

SEQ ID NO: 1 - DNA, 2916 bp, synthetic construct
  misc_feature 1..2916: note = Plasmid Sequence 1
  source 1..2916: mol_type = other DNA
atgctgaacggcgactggaaggccaaggtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcaccggcctgttcggcgccctgctggccgacaagaagaccgaggagaccaccctgctggaggaccgcatcctgaccacccgcaacggccacaccaccagcaccacccagagcagcgtgggcgtgacctacggctacagcaccaccgaggaccacgtggccggccccaacaccagcggcctggagacccgcgtggtgcaggccgagcgcttcttcaagaagttcctgttcgactggaccaccgacaagcccttcggccacctgcacaagctggagctgcccaccgaccaccacggcgtgttcggccacctggtggacagctacgcctacatgcgcaacggctgggacgtggaggtgagcgccgtgggcaaccagttcaacggcggctgcctgctggtggccatggtgcccgagtggaaggagttcgacacccgcgagaagtaccagctgaccctgttcccccaccagttcatcagcccccgcaccaacatgaccgcccacatcaccgtgccctacctgggcgtgaaccgctacgaccagtacaagaagcacaagccctggaccctggtggtgatggtggtgagccccctgaccgtgaacaccgccgcccagatcaaggtgtacgccaacatcgcccccacctacgtgcacgtggccggcgagctgcccagcaaggagggcatcttccccgtggcctgcgccgacggctacggcggcctggtgaccaccgaccccaagaccgccgaccccgcctacggcaaggtgtacaaccccccccgcaccaactaccccggccgcttcaccaacctgctggacgtggccgaggcctgccccaccttcctgtgcttcgacgacggcaagccctacgtgaccacccgcaccgacgagacccgcctgctggccaagttcgacgtgagcctggccgccaagcacatgagcaacacctacctgagcggcatcgcccagtactacacccagtacagcggcaccatcaacctgcacttcatgttcaccggcagcaccgacagcaaggcccgctacatggtggcctacatcccccccggcgtggagaccccccccgacacccccgagcgcgccgcccactgcatccacgccgagtgggacaccggcctgaacagcaagttcaccttcagcatcccctacgtgagcgccgccgactacgcctacaccgccagcgacaccgccgagaccaccaacgtgcagggctgggtgtgcgtgtaccagatcacccacggcaaggccgagaacgacaccctggtggtgagcgtgagcgccggcaaggacttcgagctgcgcctgcccatcgacccccgccagcagaccaccgccaccggcgagagcgccgaccccgtgaccaccaccgtggagaactacggcggcgagacccaggtgcagcgccgccaccacaccgacgtgggcttcatcatggaccgcttcgtgaagatcaacagccccaagcccacccacgtgatcgacctgatgcagacccaccagcacggcctggtgggcgccctgctgcgcgccgccacctactacttcagcgacctggagatcgtggtgcgccacgacggcctgacctgggtgcccaacggcgcccccgagagcgccctgagcaacaccagcaaccccaccgcctacaacaaggcccccttcacccgcctggccctgccctacaccgccccccaccgcgtgctggccaccgtgtacaacggcaccagcaagtacaccgtgagcggcagcagccgccgcggcgacctgggcagcctggccgcccgcgtggccaagcagctgcccgccagcttcaactacggcgccatcaaggccgacaccatccacgagctgctggtgcgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 2 - protein, 971 aa, synthetic construct
  REGION 1..971: note = consensus VP1-VP4 subtype A and consensus C3
MLNGDWKAKVQRKLKGAGQSSPATGSQNQSGNTGSIINNYYMQQYQNSMDTQLGDNAISGGSNEGSTDTTSTHTTNTQNNDWFSKLASSAFTGLFGALLADKKTEETTLLEDRILTTRNGHTTSTTQSSVGVTYGYSTTEDHVAGPNTSGLETRVVQAERFFKKFLFDWTTDKPFGHLHKLELPTDHHGVFGHLVDSYAYMRNGWDVEVSAVGNQFNGGCLLVAMVPEWKEFDTREKYQLTLFPHQFISPRTNMTAHITVPYLGVNRYDQYKKHKPWTLVVMVVSPLTVNTAAQIKVYANIAPTYVHVAGELPSKEGIFPVACADGYGGLVTTDPKTADPAYGKVYNPPRTNYPGRFTNLLDVAEACPTFLCFDDGKPYVTTRTDETRLLAKFDVSLAAKHMSNTYLSGIAQYYTQYSGTINLHFMFTGSTDSKARYMVAYIPPGVETPPDTPERAAHCIHAEWDTGLNSKFTFSIPYVSAADYAYTASDTAETTNVQGWVCVYQITHGKAENDTLVVSVSAGKDFELRLPIDPRQQTTATGESADPVTTTVENYGGETQVQRRHHTDVGFIMDRFVKINSPKPTHVIDLMQTHQHGLVGALLRAATYYFSDLEIVVRHDGLTWVPNGAPESALSNTSNPTAYNKAPFTRLALPYTAPHRVLATVYNGTSKYTVSGSSRRGDLGSLAARVAKQLPASFNYGAIKADTIHELLVRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 3 - DNA, 2916 bp, synthetic construct
  misc_feature 1..2916: note = plasmid 2 consensus VP1-VP4 subtype Asia 1 and consensus C3
  source 1..2916: mol_type = other DNA
atgctgaacggcgagtggaaggccaaggtgcagaagcgcctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagccgcctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccgtggccgaggacgccgtgagcggccccaacaccagcggcctggagacccgcgtgcagcaggccgagcgcttcttcaagaagcacctgttcgactggacccccaacctggccttcggccactgccactacctggagctgcccaccgagcacaagggcgtgtacggcagcctgatggacagctacgcctacatgcgcaacggctgggacatcgaggtgaccgccgtgggcaaccagttcaacggcggctgcctgctggtggccctggtgcccgagctgaagagcctggacacccgccagaagtaccagctgaccctgttcccccaccagttcatcaacccccgcaccaacatgaccgcccacatcaacgtgcccttcgtgggcgtgaaccgctacgaccagtacgccctgcacaagccctggaccctggtggtgatggtggtggcccccctgaccgtgaagaccggcggcagcgagcagatcaaggtgtacatgaacgccgcccccacctacgtgcacgtggccggcgagctgcccagcaaggagggcatcgtgcccgtggcctgcgccgacggctacggcaacatggtgaccaccgaccccaagaccgccgaccccgtgtacggcaaggtgttcaaccccccccgcaccaacctgcccggccgcttcaccaacttcctggacgtggccgaggcctgccccaccttcctgcgcttcggcgaggtgcccttcgtgaagaccgtgaacagcggcgaccgcctgctggccaagttcgacgtgagcctggccgccggccacatgagcaacacctacctggccggcctggcccagtactacacccagtacagcggcaccatgaacgtgcacttcatgttcaccggccccaccgacgccaaggcccgctacatggtggcctacatcccccccggcatgaccccccccaccgaccccgagcgcgccgcccactgcatccacagcgagtgggacaccggcctgaacagcaagttcaccttcagcatcccctacctgagcgccgccgactacgcctacaccgccagcgacaccgccgagaccaccagcgtgcagggctgggtgtgcatctaccagatcacccacggcaaggccgagggcgacgccctggtggtgagcgtgagcgccggcaaggacttcgagttccgcctgcccgtggacgcccgccgccagaccaccaccaccggcgagagcgccgaccccgtgaccaccaccgtggagaactacggcggcgagacccagaccgcccgccgcctgcacaccgacgtggccttcgtgctggaccgcttcgtgaagctgaccgcccccaagaacacccagaccctggacctgatgcagatccccagccacaccctggtgggcgccctgctgcgcagcgccacctactacttcagcgacctggaggtggccctggtgcacaccggccccgtgacctgggtgcccaacggcagccccaaggacgccctggacaaccagaccaaccccaccgcctaccagaagcagcccatcacccgcctggccctgccctacaccgccccccaccgcgtgctggccaccgtgtacaacggcaagaccacctacggcgagacccccagccgccgcggcgacatggccgccctggcccagcgcctgagcgagcgcctgcccaccagcttcaactacggcgccgtgaaggccgagaccatcaccgagctgctgatccgcatgaagcgcgccgagacctactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 4 - protein, 971 aa, synthetic construct
  REGION 1..971: note = consensus VP1-VP4 subtype Asia 1 and consensus C3
MLNGEWKAKVQKRLKGAGQSSPATGSQNQSGNTGSIINNYYMQQYQNSMDTQLGDNAISGGSNEGSTDTTSTHTNNTQNNDWFSRLASSAFSGLFGALLADKKTEETTLLEDRILTTRNGHTTSTTQSSVGVTYGYAVAEDAVSGPNTSGLETRVQQAERFFKKHLFDWTPNLAFGHCHYLELPTEHKGVYGSLMDSYAYMRNGWDIEVTAVGNQFNGGCLLVALVPELKSLDTRQKYQLTLFPHQFINPRTNMTAHINVPFVGVNRYDQYALHKPWTLVVMVVAPLTVKTGGSEQIKVYMNAAPTYVHVAGELPSKEGIVPVACADGYGNMVTTDPKTADPVYGKVFNPPRTNLPGRFTNFLDVAEACPTFLRFGEVPFVKTVNSGDRLLAKFDVSLAAGHMSNTYLAGLAQYYTQYSGTMNVHFMFTGPTDAKARYMVAYIPPGMTPPTDPERAAHCIHSEWDTGLNSKFTFSIPYLSAADYAYTASDTAETTSVQGWVCIYQITHGKAEGDALVVSVSAGKDFEFRLPVDARRQTTTTGESADPVTTTVENYGGETQTARRLHTDVAFVLDRFVKLTAPKNTQTLDLMQIPSHTLVGALLRSATYYFSDLEVALVHTGPVTWVPNGSPKDALDNQTNPTAYQKQPITRLALPYTAPHRVLATVYNGKTTYGETPSRRGDMAALAQRLSERLPTSFNYGAVKAETITELLIRMKRAETYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 5 - DNA, 2913 bp, synthetic construct
  misc_feature 1..2913: note = plasmid 3 consensus VP1-VP4 subtype C and consensus C3
  source 1..2913: mol_type = other DNA
atgctgaacgagggctggaaggccagcgtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgaccttcggctacgccaccgccgaggacagcaccagcggccccaacaccagcggcctggagacccgcgtgcaccaggccgagcgcttcttcaagatggccctgttcgactgggtgcccagccagaacttcggccacatgcacaaggtggtgctgccccacgagcccaagggcgtgtacggcggcctggtgaagagctacgcctacatgcgcaacggctgggacgtggaggtgaccgccgtgggcaaccagttcaacggcggctgcctgctggtggccctggtgcccgagatgggcgacatcagcgaccgcgagaagtaccagctgaccctgtacccccaccagttcatcaacccccgcaccaacatgaccgcccacatcaccgtgccctacgtgggcgtgaaccgctacgaccagtacaagcagcaccgcccctggaccctggtggtgatggtggtggcccccctgaccaccaacaccgccggcgcccagcagatcaaggtgtacgccaacatcgcccccaccaacgtgcacgtggccggcgagctgcccagcaaggagggcatcttccccgtggcctgcagcgacggctacggcaacatggtgaccaccgaccccaagaccgccgaccccgtgtacggcaaggtgtacaaccccccccgcaccgccctgcccggccgcttcaccaactacctggacgtggccgaggcctgccccaccttcctgatgttcgagaacgtgccctacgtgagcacccgcaccgacggccagcgcctgctggccaagttcgacgtgagcctggccgccaagcacatgagcaacacctacctggccggcctggcccagtactacacccagtacaccggcaccatcaacctgcacttcatgttcaccggccccaccgacgccaaggcccgctacatggtggcctacgtgccccccggcatggacgcccccgacaaccccgaggaggccgcccactgcatccacgccgagtgggacaccggcctgaacagcaagttcaccttcagcatcccctacatcagcgccgccgactacgcctacaccgccagccacaaggccgagaccacctgcgtgcagggctgggtgtgcgtgtaccagatcacccacggcaaggccgacgccgacgccctggtggtgagcgccagcgccggcaaggacttcgagctgcgcctgcccgtggacgcccgcaagcagaccaccaccaccggcgagagcgccgaccccgtgaccaccaccgtggagaactacggcggcgagacccaggtgcagcgccgccaccacaccgacgtggccttcgtgctggaccgcttcgtggaggtgaccgtgagcggccgcaaccagcacaccctggacgtgatgcaggcccacaaggacaacatcgtgggcgccctgctgcgcgccgccacctactacttcagcgacctggagatcgccgtgacccacaccggcaagctgacctgggtgcccaacggcgcccccgtgagcgccctgaacaacaccaccaaccccaccgcctaccacaagggccccgtgacccgcctggccctgccctacaccgccccccaccgcgtgctggccaccgcctacaccggcaccaccacctacaccgccagcgcccgcggcgacctggcccacctgaccaccacccacgcccgccacctgcccaccagcttcaacttcggcgccgtgaaggccgagaccatcaccgagctgctggtgcgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 6 - protein, 970 aa, synthetic construct
  REGION 1..970: note = Consensus VP1-VP4 subtype C and consensus C3
MLNEGWKASVQRKLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTFGYATAEDSTSGPNTSGLETRVHQAERFFKMALFDWVPSQNFGHMHKVVLPHEPKGVYGGLVKSYAYMRNGWDVEVTAVGNQFNGGCLLVALVPEMGDISDREKYQLTLYPHQFINPRTNMTAHITVPYVGVNRYDQYKQHRPWTLVVMVVAPLTTNTAGAQQIKVYANIAPTNVHVAGELPSKEGIFPVACSDGYGNMVTTDPKTADPVYGKVYNPPRTALPGRFTNYLDVAEACPTFLMFENVPYVSTRTDGQRLLAKFDVSLAAKHMSNTYLAGLAQYYTQYTGTINLHFMFTGPTDAKARYMVAYVPPGMDAPDNPEEAAHCIHAEWDTGLNSKFTFSIPYISAADYAYTASHKAETTCVQGWVCVYQITHGKADADALVVSASAGKDFELRLPVDARKQTTTTGESADPVTTTVENYGGETQVQRRHHTDVAFVLDRFVEVTVSGRNQHTLDVMQAHKDNIVGALLRAATYYFSDLEIAVTHTGKLTWVPNGAPVSALNNTTNPTAYHKGPVTRLALPYTAPHRVLATAYTGTTTYTASARGDLAHLTTTHARHLPTSFNFGAVKAETITELLVRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 7 - DNA, 2925 bp, synthetic construct
  misc_feature 1..2925: note = plasmid 4 consensus VP1-VP4 subtype O and consensus C3
  source 1..2925: mol_type = other DNA
atgctgaacggcgagtggaaggccaaggtgcagaagcgcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccaccgccgaggacttcgtgagcggccccaacaccagcggcctggagacccgcgtggtgcaggccgagcgcttcttcaagacccacctgttcgactgggtgaccagcgaccccttcggccgctgctacctgctggagctgcccaccgaccacaagggcgtgtacggcagcctgaccgacagctacgcctacatgcgcaacggctgggacgtggaggtgaccgccgtgggcaaccagttcaacggcggctgcctgctggtggccatggtgcccgagctgtgcagcatcgacaagcgcgagctgtaccagctgaccctgttcccccaccagttcatcaacccccgcaccaacatgaccgcccacatcaccgtgcccttcgtgggcgtgaaccgctacgaccagtacaaggtgcacaagccctggaccctggtggtgatggtggtggcccccctgaccgtgaacaccgagggcgccccccagatcaaggtgtacgccaacatcgcccccaccaacgtgcacgtggccggcgagttccccagcaaggagggcatcttccccgtggcctgcagcgacggctacggcggcctggtgaccaccgaccccaagaccgccgaccccgcctacggcaaggtgttcaaccccccccgcaacatgctgcccggccgcttcaccaacttcctggacgtggccgaggcctgccccaccttcctgcacttcgagggcggcgtgccctacgtgaccaccaagaccgacagcgaccgcgtgctggcccagttcgacctgagcctggccgccaagcacatgagcaacaccttcctggccggcctggcccagtactacacccagtacagcggcaccatcaacctgcacttcatgttcaccggccccaccgacgccaaggcccgctacatgatcgcctacgccccccccggcatggagccccccaagacccccgaggccgccgcccactgcatccacgccgagtgggacaccggcctgaacagcaagttcaccttcagcatcccctacctgagcgccgccgactacgcctacaccgccagcgacgccgccgagaccaccaacgtgcagggctgggtgtgcctgttccagatcacccacggcaaggccgacggcgacgccctggtggtgctggccagcgccggcaaggacttcgagctgcgcctgcccgtggacgcccgcacccagaccaccagcgccggcgagagcgccgaccccgtgaccgccaccgtggagaactacggcggcgagacccaggtgcagcgccgccagcacaccgacgtgagcttcatcctggaccgcttcgtgaaggtgacccccaaggaccagatcaacgtgctggacctgatgcagacccccgcccacaccctggtgggcgccctgctgcgcaccgccacctactacttcgccgacctggaggtggccgtgaagcacgagggcaacctgacctgggtgcccaacggcgcccccgagaccgccctggacaacaccaccaaccccaccgcctaccacaaggcccccctgacccgcctggccctgccctacaccgccccccaccgcgtgctggccaccgtgtacaacggcaactgcaagtacggcgagagccccgtgaccaacgtgcgcggcgacctgcaggtgctggcccagaaggccgcccgcaccctgcccaccagcttcaactacggcgccatcaaggccacccgcgtgaccgagctgctgtaccgcatgaagcgcgccgagacctactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 8 - protein, 974 aa, synthetic construct
  REGION 1..974: note = consensus VP1-VP4 subtype O and consensus C3
MLNGEWKAKVQKRLRGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKEGIFPVACSDGYGGLVTTDPKTADPAYGKVFNPPRNMLPGRFTNFLDVAEACPTFLHFEGGVPYVTTKTDSDRVLAQFDLSLAAKHMSNTFLAGLAQYYTQYSGTINLHFMFTGPTDAKARYMIAYAPPGMEPPKTPEAAAHCIHAEWDTGLNSKFTFSIPYLSAADYAYTASDAAETTNVQGWVCLFQITHGKADGDALVVLASAGKDFELRLPVDARTQTTSAGESADPVTATVENYGGETQVQRRQHTDVSFILDRFVKVTPKDQINVLDLMQTPAHTLVGALLRTATYYFADLEVAVKHEGNLTWVPNGAPETALDNTTNPTAYHKAPLTRLALPYTAPHRVLATVYNGNCKYGESPVTNVRGDLQVLAQKAARTLPTSFNYGAIKATRVTELLYRMKRAETYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 9 - DNA, 2952 bp, synthetic construct
  misc_feature 1..2952: note = nucleic acid, consensus VP1-VP4 subtype SAT1 and consensus C3
  source 1..2952: mol_type = other DNA
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccttcagcggcctggtgggcgccctgctggccgacaagaagaccgaggagaccaccctgctggaggaccgcatcatgaccaccagccacggcaccaccaccagcaccacccagagcagcgtgggcgtgacctacggctacgccctggccgacaagttcctgcccggccccaacaccaacggcctggagacccgcgtggagcaggccgagcgcttcttcaagcacaagctgttcgactggaccaccgaccagcagttcggcaccacccacgtgctggagctgcccaccgaccacaagggcatctacggccagctggtggacagccacgcctacatccgcaacggctgggacgtgcaggtgagcgccaccgccacccagttcaacggcggctgcctgctggtggccatggtgcccgagctgtgcaagctggacgaccgcgagaagtaccagctgaccctgttcccccaccagttcctgaacccccgcaccaacaccaccgcccacatccaggtgccctacctgggcgtggaccgccacgaccagggcacccgccacaaggcctggaccctggtggtgatggtggtggccccctacaccaacgaccagaccatcggcagcaccaaggccgaggtgtacgtgaacatcgcccccaccaacgtgtacgtggccggcgagaagcccgccaagcagggcatcctgcccgtggccgtgagcgacggctacggcggcttccagaacaccgaccccaagaccagcgaccccatctacggccacgtgtacaaccccgcccgcaccctgtaccccggccgcttcaccaacctgctggacgtggccgaggcctgccccaccctgctggacttcaacggcgtgccctacgtgcagacccagaacaacagcggcagcaaggtgctggcccgcttcgacctggccttcggccacaagaacatgaagaacacctacatgagcggcctggcccagtacttcgcccagtacagcggcaccctgaacctgcacttcatgtacaccggccccaccaacaacaaggccaagtacatggtggcctacatcccccccggcacccaccccctgcccgagacccccgagatggccagccactgctaccacgccgagtgggacaccggcctgaacagcaccttcaccttcaccgtgccctacatcagcgccgccgactacgcctacacctacgccgacgagcccgagcaggccagcgtgcagggctgggtgggcgtgtaccagatcaccgacacccacgagaaggacggcgccgtgatcgtgaccgtgagcgccggccccgacttcgagttccgcatgcccatcagccccagccgccagaccaccagcgccggcgagggcgccgaccccgtgaccaccgacgtgagcgagcacggcggcgacagccgcaccgcccgccgcgcccacaccgacgtggccttcctgctggaccgcttcaccctggtgggcaagacccaggacaacaagctggtgctggacctgctgaccaccaaggagaagagcctggtgggcgccctgctgcgcgccgccacctactacttcagcgacctggaggtggcctgcgtgggcaccaacaagtgggtgggctggacccccaacggcagccccgtgaagctgagcgaggtgggcgacaaccccgtggtgttcagccacaacggcaccacccgcttcgccctgccctacaccgccccccaccgcgtgctggccaccgtgtacaacggcgactgcaagtacaagcccaccggcacccccccccgcgagaacatccgcggcgacctggccaccctggccgcccgcatcgccagcgagacccacatccccaccaccttcaactacggcatgatctacaccgaggccgaggtggacgtgtacctgcgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 10 - protein, 983 aa, synthetic construct
  REGION 1..983: note = conconsensus VP1-VP4 subtypeSAT1 and consensus C3
MLDVDWQDRAGLFLRGAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRIMTTSHGTTTSTTQSSVGVTYGYALADKFLPGPNTNGLETRVEQAERFFKHKLFDWTTDQQFGTTHVLELPTDHKGIYGQLVDSHAYIRNGWDVQVSATATQFNGGCLLVAMVPELCKLDDREKYQLTLFPHQFLNPRTNTTAHIQVPYLGVDRHDQGTRHKAWTLVVMVVAPYTNDQTIGSTKAEVYVNIAPTNVYVAGEKPAKQGILPVAVSDGYGGFQNTDPKTSDPIYGHVYNPARTLYPGRFTNLLDVAEACPTLLDFNGVPYVQTQNNSGSKVLARFDLAFGHKNMKNTYMSGLAQYFAQYSGTLNLHFMYTGPTNNKAKYMVAYIPPGTHPLPETPEMASHCYHAEWDTGLNSTFTFTVPYISAADYAYTYADEPEQASVQGWVGVYQITDTHEKDGAVIVTVSAGPDFEFRMPISPSRQTTSAGEGADPVTTDVSEHGGDSRTARRAHTDVAFLLDRFTLVGKTQDNKLVLDLLTTKEKSLVGALLRAATYYFSDLEVACVGTNKWVGWTPNGSPVKLSEVGDNPVVFSHNGTTRFALPYTAPHRVLATVYNGDCKYKPTGTPPRENIRGDLATLAARIASETHIPTTFNYGMIYTEAEVDVYLRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 11 - DNA, 2940 bp, synthetic construct
  misc_feature 1..2940: note = nucleic acid, consensus VP1-VP4 subtype SAT2 and consensus C3
  source 1..2940: mol_type = other DNA
atgctggacgtggactggcaggacaaggccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccaccctgctggaggaccgcatcctgaccacccgccacggcaccaccaccagcaccacccagagcagcgtgggcatcacctacggctacgccgacgccgacagcttccgccccggccccaacaccagcggcctggagacccgcgtggagcaggccgagcgcttcttcaaggagaagctgttcgactggaccagcgacaagcccttcggcaccctgtacgtgctggagctgcccaaggaccacaagggcatctacggcagcctgaccgacgcctacacctacatgcgcaacggctgggacgtgcaggtgagcgccaccagcacccagttcaacggcggcagcctgctggtggccatggtgcccgagctgtgcagcctgaaggaccgcgaggagttccagctgaccctgtacccccaccagttcatcaacccccgcaccaacaccaccgcccacatccaggtgccctacctgggcgtgaaccgccacgaccagggcaagcgccaccaggcctggagcctggtggtgatggtgctgacccccctgaccaccgaggcccagatgaacagcggcaccgtggaggtgtacgccaacatcgcccccaccaacgtgttcgtggccggcgagaagcccgccaagcagggcatcatccccgtggcctgcgccgacggctacggcggcttccagaacaccgaccccaagaccgccgaccccatctacggctacgtgtacaaccccagccgcaacgactgccacggccgctacagcaacctgctggacgtggccgaggcctgccccaccctgctgaacttcgacggcaagccctacgtggtgaccaagaacaacggcgacaaggtgatggccgccttcgacgtggccttcacccacaaggtgcacaagaacaccttcctggccggcctggccgactactacacccagtaccagggcagcctgaactaccacttcatgtacaccggccccacccaccacaaggccaagttcatggtggcctacatcccccccggcatcgagaccgacaagctgcccaagacccccgaggacgccgcccactgctaccacagcgagtgggacaccggcctgaacagccagttcaccttcgccgtgccctacgtgagcgccagcgacttcagctacacccacaccgacacccccgccatggccaccaccaacggctgggtggccgtgttccaggtgaccgacacccacagcgccgaggccgccgtggtggtgagcgtgagcgccggccccgacctggagttccgcttccccatcgaccccgtgcgccagaccaccagcgccggcgagggcgccgaggtggtgaccaccgaccccagcacccacggcggcaaggtgaccgagaagcgccgcgtgcacaccgacgtggccttcgtgctggaccgcttcacccacgtgcacaccaacaagaccaccttcgccgtggacctgatggacaccaaggagaagaccctggtgggcgccctgctgcgcgccgccacctactacttctgcgacctggagatcgcctgcgtgggcgagcacaagcgcgtgttctggcagcccaacggcgccccccgcaccacccagctgggcgacaaccccatggtgttcagccacaacaaggtgacccgcttcgccatcccctacaccgccccccaccgcctgctgagcaccgtgtacaacggcgagtgcgagtacaccaagaccgtgaccgccatccgcggcgaccgcgaggtgctggccgccaagtacagcagcgccaagcacaccctgcccagcaccttcaacttcggcttcgtgaccgccgacgagcccgtggacgtgtactaccgcatgaagcgcgccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 12 - protein, 979 aa, synthetic construct
  REGION 1..979: note = consensus VP1-VP4 subtype SAT2 and consensus C3
MLDVDWQDKAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRILTTRHGTTTSTTQSSVGITYGYADADSFRPGPNTSGLETRVEQAERFFKEKLFDWTSDKPFGTLYVLELPKDHKGIYGSLTDAYTYMRNGWDVQVSATSTQFNGGSLLVAMVPELCSLKDREEFQLTLYPHQFINPRTNTTAHIQVPYLGVNRHDQGKRHQAWSLVVMVLTPLTTEAQMNSGTVEVYANIAPTNVFVAGEKPAKQGIIPVACADGYGGFQNTDPKTADPIYGYVYNPSRNDCHGRYSNLLDVAEACPTLLNFDGKPYVVTKNNGDKVMAAFDVAFTHKVHKNTFLAGLADYYTQYQGSLNYHFMYTGPTHHKAKFMVAYIPPGIETDKLPKTPEDAAHCYHSEWDTGLNSQFTFAVPYVSASDFSYTHTDTPAMATTNGWVAVFQVTDTHSAEAAVVVSVSAGPDLEFRFPIDPVRQTTSAGEGAEVVTTDPSTHGGKVTEKRRVHTDVAFVLDRFTHVHTNKTTFAVDLMDTKEKTLVGALLRAATYYFCDLEIACVGEHKRVFWQPNGAPRTTQLGDNPMVFSHNKVTRFAIPYTAPHRLLSTVYNGECEYTKTVTAIRGDREVLAAKYSSAKHTLPSTFNFGFVTADEPVDVYYRMKRAELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 13 - DNA, 2937 bp, synthetic construct
  misc_feature 1..2937: note = nucleic acid, consensus VP1-VP4 subtype SAT3 and consensus C3
  source 1..2937: mol_type = other DNA
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccacccacctggaggaccgcatcctgaccacccgccacaacaccaccaccagcaccacccagagcagcgtgggcgtgacctacggctacgtgagcgccgaccgcttcctgcccggccccaacaccagcggcctggagagccgcgtggagcaggccgagcgcttcttcaaggagaagctgttcacctggaccgccagccaggagtacgcccacgtgcacctgctggagctgcccaccgaccacaagggcatctacggcgccatggtggacagccacgcctacgtgcgcaacggctgggacgtgcaggtgaccgccaccagcacccagttcaacggcggcaccctgctggtggccatggtgcccgagctgcacagcctggacacccgcgacgtgagccagctgaccctgttcccccaccagttcatcaacccccgcaccaacaccaccgcccacatcgtggtgccctacgtgggcgtgaaccgccacgaccaggtgcagatgcacaaggcctggaccctggtggtggccgtgatggcccccctgaccaccagcagcatgggccaggacaacgtggaggtgtacgccaacatcgcccccaccaacgtgtacgtggccggcgagcgccccagcaagcagggcatcatccccgtggcctgcaacgacggctacggcggcttccagaacaccgaccccaagaccgccgaccccatctacggcctggtgagcaaccccccccgcaccgccttccccggccgcttcaccaacctgctggacgtggccgaggcctgccccaccttcctggacttcgacggcgtgccctacgtgaagaccacccacaacagcggcagcaagatcctgacccacatcgacctggccttcggccacaagagcttcaagaacacctacctggccggcctggcccagtactacgcccagtacagcggcagcatcaacctgcacttcatgtacaccggccccacccagagcaaggcccgcttcatggtggcctacatcccccccggcaccaccgtgcccaacacccccgagcaggccgcccactgctaccacagcgagtgggacaccggcctgaacagcaagttcaccttcaccgtgccctacatgagcgccgccgacttcgcctacacctactgcgacgagcccgagcaggccagcgcccagggctgggtgaccctgtaccagatcaccgacacccacgaccccgacagcgccgtgctggtgagcgtgagcgccggcgccgacttcgagctgcgcctgcccatcaaccccgccgcccagaccaccagcgccggcgagggcgccgacgtggtgaccaccgacgtgaccacccacggcggcgaggtgagcgtgccccgccgccagcacaccaacgtggagttcctgctggaccgcttcacccacatcggcaccatcaacggccaccgcaccatctgcctgatggacaccaaggagcacaccctggtgggcgccatcctgcgcagcgccacctactacttctgcgacctggaggtggccgtgctgggcaacgccaagtacgccgcctgggtgcccaacggctgcccccacaccgaccgcgtggaggacaaccccgtggtgcacagcaagggcagcgtggtgcgcttcgccctgccctacaccgccccccacggcgtgctggccaccgtgtacaacggcaactgcaagtacagcaccacccagcgcgtggccccccgccgcggcgacctgggcgtgctgagccagcgcgtggagaacgagaccacccgctgcatccccaccaccttcaacttcggccgcctgctgtgcgagagcggcgacgtgtactaccgcatgaagcgcaccgagctgtactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 14 - protein, 978 aa, synthetic construct
  REGION 1..978: note = consensus VP1-VP4 subtype SAT3 and consensusC3
MLDVDWQDRAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTHLEDRILTTRHNTTTSTTQSSVGVTYGYVSADRFLPGPNTSGLESRVEQAERFFKEKLFTWTASQEYAHVHLLELPTDHKGIYGAMVDSHAYVRNGWDVQVTATSTQFNGGTLLVAMVPELHSLDTRDVSQLTLFPHQFINPRTNTTAHIVVPYVGVNRHDQVQMHKAWTLVVAVMAPLTTSSMGQDNVEVYANIAPTNVYVAGERPSKQGIIPVACNDGYGGFQNTDPKTADPIYGLVSNPPRTAFPGRFTNLLDVAEACPTFLDFDGVPYVKTTHNSGSKILTHIDLAFGHKSFKNTYLAGLAQYYAQYSGSINLHFMYTGPTQSKARFMVAYIPPGTTVPNTPEQAAHCYHSEWDTGLNSKFTFTVPYMSAADFAYTYCDEPEQASAQGWVTLYQITDTHDPDSAVLVSVSAGADFELRLPINPAAQTTSAGEGADVVTTDVTTHGGEVSVPRRQHTNVEFLLDRFTHIGTINGHRTICLMDTKEHTLVGAILRSATYYFCDLEVAVLGNAKYAAWVPNGCPHTDRVEDNPVVHSKGSVVRFALPYTAPHGVLATVYNGNCKYSTTQRVAPRRGDLGVLSQRVENETTRCIPTTFNFGRLLCESGDVYYRMKRTELYCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 15 - DNA, 756 bp, synthetic construct
  misc_feature 1..756: note = consensus FMDV Protease c3 sequence
  source 1..756: mol_type = other DNA
tactgcgtgaagaagcccgtggccctgaaggtgaaggccaagaacaccctgatcgtgaccgagagcggcgccccccccaccgacctgcagaagatggtgatgggcaacaccaagcccgtggagctgatcctggacggcaagaccgtggccatctgctgcgccaccggcgtgttcggcaccgcctacctggtgccccgccacctgttcgccgagaagtacgacaagatcatgctggacggccgcgccatgaccgacagcgactaccgcgtgttcgagttcgagatcaaggtgaagggccaggacatgctgagcgacgccgccctgatggtgctgcaccgcggcaaccgcgtgcgcgacatcaccaagcacttccgcgacaccgcccgcatgaagaagggcacccccgtggtgggcgtgatcaacaacgccgacgtgggccgcctgatcttcagcggcgaggccctgacctacaaggacatcgtggtgtgcatggacggcgacaccatgcccggcctgttcgcctacaaggccgccaccaaggccggctactgcggcggcgccgtgctggccaaggacggcgccgacaccttcatcgtgggcacccacagcgccggcggccgcaacggcgtgggctactgcagctgcgtgagccgcagcatgctgctgaagatgaaggcccacatcgaccccgagccccaccacgagggcctgatcgtggacacccgcgacgtggaggagcgcgtgcacgtgatgtga

SEQ ID NO: 16 - protein, 251 aa, synthetic construct
  REGION 1..251: note = Amino Acid consensus Protease c3 sequence
YCVKKPVALKVKAKNTLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGRNGVGYCSCVSRSMLLKMKAHIDPEPHHEGLIVDTRDVEERVHVM

SEQ ID NO: 17 - DNA, 2160 bp, synthetic construct
  misc_feature 1..2160: note = nucleic acid, consensus vp1-4 Subtype A
  source 1..2160: mol_type = other DNA
atgctgaacggcgactggaaggccaaggtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcaccggcctgttcggcgccctgctggccgacaagaagaccgaggagaccaccctgctggaggaccgcatcctgaccacccgcaacggccacaccaccagcaccacccagagcagcgtgggcgtgacctacggctacagcaccaccgaggaccacgtggccggccccaacaccagcggcctggagacccgcgtggtgcaggccgagcgcttcttcaagaagttcctgttcgactggaccaccgacaagcccttcggccacctgcacaagctggagctgcccaccgaccaccacggcgtgttcggccacctggtggacagctacgcctacatgcgcaacggctgggacgtggaggtgagcgccgtgggcaaccagttcaacggcggctgcctgctggtggccatggtgcccgagtggaaggagttcgacacccgcgagaagtaccagctgaccctgttcccccaccagttcatcagcccccgcaccaacatgaccgcccacatcaccgtgccctacctgggcgtgaaccgctacgaccagtacaagaagcacaagccctggaccctggtggtgatggtggtgagccccctgaccgtgaacaccgccgcccagatcaaggtgtacgccaacatcgcccccacctacgtgcacgtggccggcgagctgcccagcaaggagggcatcttccccgtggcctgcgccgacggctacggcggcctggtgaccaccgaccccaagaccgccgaccccgcctacggcaaggtgtacaaccccccccgcaccaactaccccggccgcttcaccaacctgctggacgtggccgaggcctgccccaccttcctgtgcttcgacgacggcaagccctacgtgaccacccgcaccgacgagacccgcctgctggccaagttcgacgtgagcctggccgccaagcacatgagcaacacctacctgagcggcatcgcccagtactacacccagtacagcggcaccatcaacctgcacttcatgttcaccggcagcaccgacagcaaggcccgctacatggtggcctacatcccccccggcgtggagaccccccccgacacccccgagcgcgccgcccactgcatccacgccgagtgggacaccggcctgaacagcaagttcaccttcagcatcccctacgtgagcgccgccgactacgcctacaccgccagcgacaccgccgagaccaccaacgtgcagggctgggtgtgcgtgtaccagatcacccacggcaaggccgagaacgacaccctggtggtgagcgtgagcgccggcaaggacttcgagctgcgcctgcccatcgacccccgccagcagaccaccgccaccggcgagagcgccgaccccgtgaccaccaccgtggagaactacggcggcgagacccaggtgcagcgccgccaccacaccgacgtgggcttcatcatggaccgcttcgtgaagatcaacagccccaagcccacccacgtgatcgacctgatgcagacccaccagcacggcctggtgggcgccctgctgcgcgccgccacctactacttcagcgacctggagatcgtggtgcgccacgacggcctgacctgggtgcccaacggcgcccccgagagcgccctgagcaacaccagcaaccccaccgcctacaacaaggcccccttcacccgcctggccctgccctacaccgccccccaccgcgtgctggccaccgtgtacaacggcaccagcaagtacaccgtgagcggcagcagccgccgcggcgacctgggcagcctggccgcccgcgtggccaagcagctgcccgccagcttcaactacggcgccatcaaggccgacaccatccacgagctgctggtgcgcatgaagcgcgccgagctg

SEQ ID NO: 18 - protein, 720 aa, synthetic construct
  REGION 1..720: note = Consensus vp1-4 Subtype A
MLNGDWKAKVQRKLKGAGQSSPATGSQNQSGNTGSIINNYYMQQYQNSMDTQLGDNAISGGSNEGSTDTTSTHTTNTQNNDWFSKLASSAFTGLFGALLADKKTEETTLLEDRILTTRNGHTTSTTQSSVGVTYGYSTTEDHVAGPNTSGLETRVVQAERFFKKFLFDWTTDKPFGHLHKLELPTDHHGVFGHLVDSYAYMRNGWDVEVSAVGNQFNGGCLLVAMVPEWKEFDTREKYQLTLFPHQFISPRTNMTAHITVPYLGVNRYDQYKKHKPWTLVVMVVSPLTVNTAAQIKVYANIAPTYVHVAGELPSKEGIFPVACADGYGGLVTTDPKTADPAYGKVYNPPRTNYPGRFTNLLDVAEACPTFLCFDDGKPYVTTRTDETRLLAKFDVSLAAKHMSNTYLSGIAQYYTQYSGTINLHFMFTGSTDSKARYMVAYIPPGVETPPDTPERAAHCIHAEWDTGLNSKFTFSIPYVSAADYAYTASDTAETTNVQGWVCVYQITHGKAENDTLVVSVSAGKDFELRLPIDPRQQTTATGESADPVTTTVENYGGETQVQRRHHTDVGFIMDRFVKINSPKPTHVIDLMQTHQHGLVGALLRAATYYFSDLEIVVRHDGLTWVPNGAPESALSNTSNPTAYNKAPFTRLALPYTAPHRVLATVYNGTSKYTVSGSSRRGDLGSLAARVAKQLPASFNYGAIKADTIHELLVRMKRAEL

SEQ ID NO: 19 - DNA, 2160 bp, synthetic construct
  misc_feature 1..2160: note = nucleic acid, consensus vp1-4 Subtype Asia 1
  source 1..2160: mol_type = other DNA
atgctgaacggcgagtggaaggccaaggtgcagaagcgcctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagccgcctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccgtggccgaggacgccgtgagcggccccaacaccagcggcctggagacccgcgtgcagcaggccgagcgcttcttcaagaagcacctgttcgactggacccccaacctggccttcggccactgccactacctggagctgcccaccgagcacaagggcgtgtacggcagcctgatggacagctacgcctacatgcgcaacggctgggacatcgaggtgaccgccgtgggcaaccagttcaacggcggctgcctgctggtggccctggtgcccgagctgaagagcctggacacccgccagaagtaccagctgaccctgttcccccaccagttcatcaacccccgcaccaacatgaccgcccacatcaacgtgcccttcgtgggcgtgaaccgctacgaccagtacgccctgcacaagccctggaccctggtggtgatggtggtggcccccctgaccgtgaagaccggcggcagcgagcagatcaaggtgtacatgaacgccgcccccacctacgtgcacgtggccggcgagctgcccagcaaggagggcatcgtgcccgtggcctgcgccgacggctacggcaacatggtgaccaccgaccccaagaccgccgaccccgtgtacggcaaggtgttcaaccccccccgcaccaacctgcccggccgcttcaccaacttcctggacgtggccgaggcctgccccaccttcctgcgcttcggcgaggtgcccttcgtgaagaccgtgaacagcggcgaccgcctgctggccaagttcgacgtgagcctggccgccggccacatgagcaacacctacctggccggcctggcccagtactacacccagtacagcggcaccatgaacgtgcacttcatgttcaccggccccaccgacgccaaggcccgctacatggtggcctacatcccccccggcatgaccccccccaccgaccccgagcgcgccgcccactgcatccacagcgagtgggacaccggcctgaacagcaagttcaccttcagcatcccctacctgagcgccgccgactacgcctacaccgccagcgacaccgccgagaccaccagcgtgcagggctgggtgtgcatctaccagatcacccacggcaaggccgagggcgacgccctggtggtgagcgtgagcgccggcaaggacttcgagttccgcctgcccgtggacgcccgccgccagaccaccaccaccggcgagagcgccgaccccgtgaccaccaccgtggagaactacggcggcgagacccagaccgcccgccgcctgcacaccgacgtggccttcgtgctggaccgcttcgtgaagctgaccgcccccaagaacacccagaccctggacctgatgcagatccccagccacaccctggtgggcgccctgctgcgcagcgccacctactacttcagcgacctggaggtggccctggtgcacaccggccccgtgacctgggtgcccaacggcagccccaaggacgccctggacaaccagaccaaccccaccgcctaccagaagcagcccatcacccgcctggccctgccctacaccgccccccaccgcgtgctggccaccgtgtacaacggcaagaccacctacggcgagacccccagccgccgcggcgacatggccgccctggcccagcgcctgagcgagcgcctgcccaccagcttcaactacggcgccgtgaaggccgagaccatcaccgagctgctgatccgcatgaagcgcgccgagacc

SEQ ID NO: 20 - protein, 704 aa, synthetic construct
  REGION 1..704: note = Consensus vp1-4 Subtype Asia 1
AGQSSPATGSQNQSGNTGSIINNYYMQQYQNSMDTQLGDNAISGGSNEGSTDTTSTHTNNTQNNDWFSRLASSAFSGLFGALLADKKTEETTLLEDRILTTRNGHTTSTTQSSVGVTYGYAVAEDAVSGPNTSGLETRVQQAERFFKKHLFDWTPNLAFGHCHYLELPTEHKGVYGSLMDSYAYMRNGWDIEVTAVGNQFNGGCLLVALVPELKSLDTRQKYQLTLFPHQFINPRTNMTAHINVPFVGVNRYDQYALHKPWTLVVMVVAPLTVKTGGSEQIKVYMNAAPTYVHVAGELPSKEGIVPVACADGYGNMVTTDPKTADPVYGKVFNPPRTNLPGRFTNFLDVAEACPTFLRFGEVPFVKTVNSGDRLLAKFDVSLAAGHMSNTYLAGLAQYYTQYSGTMNVHFMFTGPTDAKARYMVAYIPPGMTPPTDPERAAHCIHSEWDTGLNSKFTFSIPYLSAADYAYTASDTAETTSVQGWVCIYQITHGKAEGDALVVSVSAGKDFEFRLPVDARRQTTTTGESADPVTTTVENYGGETQTARRLHTDVAFVLDRFVKLTAPKNTQTLDLMQIPSHTLVGALLRSATYYFSDLEVALVHTGPVTWVPNGSPKDALDNQTNPTAYQKQPITRLALPYTAPHRVLATVYNGKTTYGETPSRRGDMAALAQRLSERLPTSFNYGAVKAETITELLIRMKRAET

SEQ ID NO: 21 - DNA, 2157 bp, synthetic construct
  misc_feature 1..2157: note = nucleic acid, consensus vp1-4 Subtype C
  source 1..2157: mol_type = other DNA
atgctgaacgagggctggaaggccagcgtgcagcgcaagctgaagggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgaccttcggctacgccaccgccgaggacagcaccagcggccccaacaccagcggcctggagacccgcgtgcaccaggccgagcgcttcttcaagatggccctgttcgactgggtgcccagccagaacttcggccacatgcacaaggtggtgctgccccacgagcccaagggcgtgtacggcggcctggtgaagagctacgcctacatgcgcaacggctgggacgtggaggtgaccgccgtgggcaaccagttcaacggcggctgcctgctggtggccctggtgcccgagatgggcgacatcagcgaccgcgagaagtaccagctgaccctgtacccccaccagttcatcaacccccgcaccaacatgaccgcccacatcaccgtgccctacgtgggcgtgaaccgctacgaccagtacaagcagcaccgcccctggaccctggtggtgatggtggtggcccccctgaccaccaacaccgccggcgcccagcagatcaaggtgtacgccaacatcgcccccaccaacgtgcacgtggccggcgagctgcccagcaaggagggcatcttccccgtggcctgcagcgacggctacggcaacatggtgaccaccgaccccaagaccgccgaccccgtgtacggcaaggtgtacaaccccccccgcaccgccctgcccggccgcttcaccaactacctggacgtggccgaggcctgccccaccttcctgatgttcgagaacgtgccctacgtgagcacccgcaccgacggccagcgcctgctggccaagttcgacgtgagcctggccgccaagcacatgagcaacacctacctggccggcctggcccagtactacacccagtacaccggcaccatcaacctgcacttcatgttcaccggccccaccgacgccaaggcccgctacatggtggcctacgtgccccccggcatggacgcccccgacaaccccgaggaggccgcccactgcatccacgccgagtgggacaccggcctgaacagcaagttcaccttcagcatcccctacatcagcgccgccgactacgcctacaccgccagccacaaggccgagaccacctgcgtgcagggctgggtgtgcgtgtaccagatcacccacggcaaggccgacgccgacgccctggtggtgagcgccagcgccggcaaggacttcgagctgcgcctgcccgtggacgcccgcaagcagaccaccaccaccggcgagagcgccgaccccgtgaccaccaccgtggagaactacggcggcgagacccaggtgcagcgccgccaccacaccgacgtggccttcgtgctggaccgcttcgtggaggtgaccgtgagcggccgcaaccagcacaccctggacgtgatgcaggcccacaaggacaacatcgtgggcgccctgctgcgcgccgccacctactacttcagcgacctggagatcgccgtgacccacaccggcaagctgacctgggtgcccaacggcgcccccgtgagcgccctgaacaacaccaccaaccccaccgcctaccacaagggccccgtgacccgcctggccctgccctacaccgccccccaccgcgtgctggccaccgcctacaccggcaccaccacctacaccgccagcgcccgcggcgacctggcccacctgaccaccacccacgcccgccacctgcccaccagcttcaacttcggcgccgtgaaggccgagaccatcaccgagctgctggtgcgcatgaagcgcgccgagctg

SEQ ID NO: 22 - protein, 719 aa, synthetic construct
  REGION 1..719: note = Consensus vp1-4 Subtype C
MLNEGWKASVQRKLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTFGYATAEDSTSGPNTSGLETRVHQAERFFKMALFDWVPSQNFGHMHKVVLPHEPKGVYGGLVKSYAYMRNGWDVEVTAVGNQFNGGCLLVALVPEMGDISDREKYQLTLYPHQFINPRTNMTAHITVPYVGVNRYDQYKQHRPWTLVVMVVAPLTTNTAGAQQIKVYANIAPTNVHVAGELPSKEGIFPVACSDGYGNMVTTDPKTADPVYGKVYNPPRTALPGRFTNYLDVAEACPTFLMFENVPYVSTRTDGQRLLAKFDVSLAAKHMSNTYLAGLAQYYTQYTGTINLHFMFTGPTDAKARYMVAYVPPGMDAPDNPEEAAHCIHAEWDTGLNSKFTFSIPYISAADYAYTASHKAETTCVQGWVCVYQITHGKADADALVVSASAGKDFELRLPVDARKQTTTTGESADPVTTTVENYGGETQVQRRHHTDVAFVLDRFVEVTVSGRNQHTLDVMQAHKDNIVGALLRAATYYFSDLEIAVTHTGKLTWVPNGAPVSALNNTTNPTAYHKGPVTRLALPYTAPHRVLATAYTGTTTYTASARGDLAHLTTTHARHLPTSFNFGAVKAETITELLVRMKRAEL

SEQ ID NO: 23 - DNA, 2169 bp, synthetic construct
  misc_feature 1..2169: note = nucleic acid, consensus vp1-4 Subtype O
  source 1..2169: mol_type = other DNA
atgctgaacggcgagtggaaggccaaggtgcagaagcgcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaccaacacccagaacaacgactggttcagcaagctggccagcagcgccttcagcggcctgttcggcgccctgctggccgacaagaagaccgaggagaccaccctgctggaggaccgcatcctgaccacccgcaacggccacaccaccagcaccacccagagcagcgtgggcgtgacctacggctacgccaccgccgaggacttcgtgagcggccccaacaccagcggcctggagacccgcgtggtgcaggccgagcgcttcttcaagacccacctgttcgactgggtgaccagcgaccccttcggccgctgctacctgctggagctgcccaccgaccacaagggcgtgtacggcagcctgaccgacagctacgcctacatgcgcaacggctgggacgtggaggtgaccgccgtgggcaaccagttcaacggcggctgcctgctggtggccatggtgcccgagctgtgcagcatcgacaagcgcgagctgtaccagctgaccctgttcccccaccagttcatcaacccccgcaccaacatgaccgcccacatcaccgtgcccttcgtgggcgtgaaccgctacgaccagtacaaggtgcacaagccctggaccctggtggtgatggtggtggcccccctgaccgtgaacaccgagggcgccccccagatcaaggtgtacgccaacatcgcccccaccaacgtgcacgtggccggcgagttccccagcaaggagggcatcttccccgtggcctgcagcgacggctacggcggcctggtgaccaccgaccccaagaccgccgaccccgcctacggcaaggtgttcaaccccccccgcaacatgctgcccggccgcttcaccaacttcctggacgtggccgaggcctgccccaccttcctgcacttcgagggcggcgtgccctacgtgaccaccaagaccgacagcgaccgcgtgctggcccagttcgacctgagcctggccgccaagcacatgagcaacaccttcctggccggcctggcccagtactacacccagtacagcggcaccatcaacctgcacttcatgttcaccggccccaccgacgccaaggcccgctacatgatcgcctacgccccccccggcatggagccccccaagacccccgaggccgccgcccactgcatccacgccgagtgggacaccggcctgaacagcaagttcaccttcagcatcccctacctgagcgccgccgactacgcctacaccgccagcgacgccgccgagaccaccaacgtgcagggctgggtgtgcctgttccagatcacccacggcaaggccgacggcgacgccctggtggtgctggccagcgccggcaaggacttcgagctgcgcctgcccgtggacgcccgcacccagaccaccagcgccggcgagagcgccgaccccgtgaccgccaccgtggagaactacggcggcgagacccaggtgcagcgccgccagcacaccgacgtgagcttcatcctggaccgcttcgtgaaggtgacccccaaggaccagatcaacgtgctggacctgatgcagacccccgcccacaccctggtgggcgccctgctgcgcaccgccacctactacttcgccgacctggaggtggccgtgaagcacgagggcaacctgacctgggtgcccaacggcgcccccgagaccgccctggacaacaccaccaaccccaccgcctaccacaaggcccccctgacccgcctggccctgccctacaccgccccccaccgcgtgctggccaccgtgtacaacggcaactgcaagtacggcgagagccccgtgaccaacgtgcgcggcgacctgcaggtgctggcccagaaggccgcccgcaccctgcccaccagcttcaactacggcgccatcaaggccacccgcgtgaccgagctgctgtaccgcatgaagcgcgccgagacc

SEQ ID NO: 24 - protein, 723 aa, synthetic construct
  REGION 1..723: note = Consensus vp1-4 Subtype O
MLNGEWKAKVQKRLRGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKEGIFPVACSDGYGGLVTTDPKTADPAYGKVFNPPRNMLPGRFTNFLDVAEACPTFLHFEGGVPYVTTKTDSDRVLAQFDLSLAAKHMSNTFLAGLAQYYTQYSGTINLHFMFTGPTDAKARYMIAYAPPGMEPPKTPEAAAHCIHAEWDTGLNSKFTFSIPYLSAADYAYTASDAAETTNVQGWVCLFQITHGKADGDALVVLASAGKDFELRLPVDARTQTTSAGESADPVTATVENYGGETQVQRRQHTDVSFILDRFVKVTPKDQINVLDLMQTPAHTLVGALLRTATYYFADLEVAVKHEGNLTWVPNGAPETALDNTTNPTAYHKAPLTRLALPYTAPHRVLATVYNGNCKYGESPVTNVRGDLQVLAQKAARTLPTSFNYGAIKATRVTELLYRMKRAET

SEQ ID NO: 25 - DNA, 2196 bp, synthetic construct
  misc_feature 1..2196: note = nucleic acid, Consensus vp1-4 Subtype SAT1
  source 1..2196: mol_type = other DNA
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccttcagcggcctggtgggcgccctgctggccgacaagaagaccgaggagaccaccctgctggaggaccgcatcatgaccaccagccacggcaccaccaccagcaccacccagagcagcgtgggcgtgacctacggctacgccctggccgacaagttcctgcccggccccaacaccaacggcctggagacccgcgtggagcaggccgagcgcttcttcaagcacaagctgttcgactggaccaccgaccagcagttcggcaccacccacgtgctggagctgcccaccgaccacaagggcatctacggccagctggtggacagccacgcctacatccgcaacggctgggacgtgcaggtgagcgccaccgccacccagttcaacggcggctgcctgctggtggccatggtgcccgagctgtgcaagctggacgaccgcgagaagtaccagctgaccctgttcccccaccagttcctgaacccccgcaccaacaccaccgcccacatccaggtgccctacctgggcgtggaccgccacgaccagggcacccgccacaaggcctggaccctggtggtgatggtggtggccccctacaccaacgaccagaccatcggcagcaccaaggccgaggtgtacgtgaacatcgcccccaccaacgtgtacgtggccggcgagaagcccgccaagcagggcatcctgcccgtggccgtgagcgacggctacggcggcttccagaacaccgaccccaagaccagcgaccccatctacggccacgtgtacaaccccgcccgcaccctgtaccccggccgcttcaccaacctgctggacgtggccgaggcctgccccaccctgctggacttcaacggcgtgccctacgtgcagacccagaacaacagcggcagcaaggtgctggcccgcttcgacctggccttcggccacaagaacatgaagaacacctacatgagcggcctggcccagtacttcgcccagtacagcggcaccctgaacctgcacttcatgtacaccggccccaccaacaacaaggccaagtacatggtggcctacatcccccccggcacccaccccctgcccgagacccccgagatggccagccactgctaccacgccgagtgggacaccggcctgaacagcaccttcaccttcaccgtgccctacatcagcgccgccgactacgcctacacctacgccgacgagcccgagcaggccagcgtgcagggctgggtgggcgtgtaccagatcaccgacacccacgagaaggacggcgccgtgatcgtgaccgtgagcgccggccccgacttcgagttccgcatgcccatcagccccagccgccagaccaccagcgccggcgagggcgccgaccccgtgaccaccgacgtgagcgagcacggcggcgacagccgcaccgcccgccgcgcccacaccgacgtggccttcctgctggaccgcttcaccctggtgggcaagacccaggacaacaagctggtgctggacctgctgaccaccaaggagaagagcctggtgggcgccctgctgcgcgccgccacctactacttcagcgacctggaggtggcctgcgtgggcaccaacaagtgggtgggctggacccccaacggcagccccgtgaagctgagcgaggtgggcgacaaccccgtggtgttcagccacaacggcaccacccgcttcgccctgccctacaccgccccccaccgcgtgctggccaccgtgtacaacggcgactgcaagtacaagcccaccggcacccccccccgcgagaacatccgcggcgacctggccaccctggccgcccgcatcgccagcgagacccacatccccaccaccttcaactacggcatgatctacaccgaggccgaggtggacgtgtacctgcgcatgaagcgcgccgagctg

SEQ ID NO: 26 - protein, 732 aa, synthetic construct
  REGION 1..732: note = Consensus vp1-4 Subtype SAT1
MLDVDWQDRAGLFLRGAGQSSPATGSQNQSGNTGSIINNYYMQQYQNSMDTQLGDNAISGGSNEGSTDTTSTHTNNTQNNDWFSKLAQSAFSGLVGALLADKKTEETTLLEDRIMTTSHGTTTSTTQSSVGVTYGYALADKFLPGPNTNGLETRVEQAERFFKHKLFDWTTDQQFGTTHVLELPTDHKGIYGQLVDSHAYIRNGWDVQVSATATQFNGGCLLVAMVPELCKLDDREKYQLTLFPHQFLNPRTNTTAHIQVPYLGVDRHDQGTRHKAWTLVVMVVAPYTNDQTIGSTKAEVYVNIAPTNVYVAGEKPAKQGILPVAVSDGYGGFQNTDPKTSDPIYGHVYNPARTLYPGRFTNLLDVAEACPTLLDFNGVPYVQTQNNSGSKVLARFDLAFGHKNMKNTYMSGLAQYFAQYSGTLNLHFMYTGPTNNKAKYMVAYIPPGTHPLPETPEMASHCYHAEWDTGLNSTFTFTVPYISAADYAYTYADEPEQASVQGWVGVYQITDTHEKDGAVIVTVSAGPDFEFRMPISPSRQTTSAGEGADPVTTDVSEHGGDSRTARRAHTDVAFLLDRFTLVGKTQDNKLVLDLLTTKEKSLVGALLRAATYYFSDLEVACVGTNKWVGWTPNGSPVKLSEVGDNPVVFSHNGTTRFALPYTAPHRVLATVYNGDCKYKPTGTPPRENIRGDLATLAARIASETHIPTTFNYGMIYTEAEVDVYLRMKRAEL

SEQ ID NO: 27 - DNA, 2184 bp, synthetic construct
  misc_feature 1..2184: note = nucleic acid, Consensus vp1-4 Subtype SAT2
  source 1..2184: mol_type = other DNA
atgctggacgtggactggcaggacaaggccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccaccctgctggaggaccgcatcctgaccacccgccacggcaccaccaccagcaccacccagagcagcgtgggcatcacctacggctacgccgacgccgacagcttccgccccggccccaacaccagcggcctggagacccgcgtggagcaggccgagcgcttcttcaaggagaagctgttcgactggaccagcgacaagcccttcggcaccctgtacgtgctggagctgcccaaggaccacaagggcatctacggcagcctgaccgacgcctacacctacatgcgcaacggctgggacgtgcaggtgagcgccaccagcacccagttcaacggcggcagcctgctggtggccatggtgcccgagctgtgcagcctgaaggaccgcgaggagttccagctgaccctgtacccccaccagttcatcaacccccgcaccaacaccaccgcccacatccaggtgccctacctgggcgtgaaccgccacgaccagggcaagcgccaccaggcctggagcctggtggtgatggtgctgacccccctgaccaccgaggcccagatgaacagcggcaccgtggaggtgtacgccaacatcgcccccaccaacgtgttcgtggccggcgagaagcccgccaagcagggcatcatccccgtggcctgcgccgacggctacggcggcttccagaacaccgaccccaagaccgccgaccccatctacggctacgtgtacaaccccagccgcaacgactgccacggccgctacagcaacctgctggacgtggccgaggcctgccccaccctgctgaacttcgacggcaagccctacgtggtgaccaagaacaacggcgacaaggtgatggccgccttcgacgtggccttcacccacaaggtgcacaagaacaccttcctggccggcctggccgactactacacccagtaccagggcagcctgaactaccacttcatgtacaccggccccacccaccacaaggccaagttcatggtggcctacatcccccccggcatcgagaccgacaagctgcccaagacccccgaggacgccgcccactgctaccacagcgagtgggacaccggcctgaacagccagttcaccttcgccgtgccctacgtgagcgccagcgacttcagctacacccacaccgacacccccgccatggccaccaccaacggctgggtggccgtgttccaggtgaccgacacccacagcgccgaggccgccgtggtggtgagcgtgagcgccggccccgacctggagttccgcttccccatcgaccccgtgcgccagaccaccagcgccggcgagggcgccgaggtggtgaccaccgaccccagcacccacggcggcaaggtgaccgagaagcgccgcgtgcacaccgacgtggccttcgtgctggaccgcttcacccacgtgcacaccaacaagaccaccttcgccgtggacctgatggacaccaaggagaagaccctggtgggcgccctgctgcgcgccgccacctactacttctgcgacctggagatcgcctgcgtgggcgagcacaagcgcgtgttctggcagcccaacggcgccccccgcaccacccagctgggcgacaaccccatggtgttcagccacaacaaggtgacccgcttcgccatcccctacaccgccccccaccgcctgctgagcaccgtgtacaacggcgagtgcgagtacaccaagaccgtgaccgccatccgcggcgaccgcgaggtgctggccgccaagtacagcagcgccaagcacaccctgcccagcaccttcaacttcggcttcgtgaccgccgacgagcccgtggacgtgtactaccgcatgaagcgcgccgagctg

SEQ ID NO: 28 - protein, 728 aa, synthetic construct
  REGION 1..728: note = Consensus vp1-4 Subtype SAT2
MLDVDWQDKAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTLLEDRILTTRHGTTTSTTQSSVGITYGYADADSFRPGPNTSGLETRVEQAERFFKEKLFDWTSDKPFGTLYVLELPKDHKGIYGSLTDAYTYMRNGWDVQVSATSTQFNGGSLLVAMVPELCSLKDREEFQLTLYPHQFINPRTNTTAHIQVPYLGVNRHDQGKRHQAWSLVVMVLTPLTTEAQMNSGTVEVYANIAPTNVFVAGEKPAKQGIIPVACADGYGGFQNTDPKTADPIYGYVYNPSRNDCHGRYSNLLDVAEACPTLLNFDGKPYVVTKNNGDKVMAAFDVAFTHKVHKNTFLAGLADYYTQYQGSLNYHFMYTGPTHHKAKFMVAYIPPGIETDKLPKTPEDAAHCYHSEWDTGLNSQFTFAVPYVSASDFSYTHTDTPAMATTNGWVAVFQVTDTHSAEAAVVVSVSAGPDLEFRFPIDPVRQTTSAGEGAEVVTTDPSTHGGKVTEKRRVHTDVAFVLDRFTHVHTNKTTFAVDLMDTKEKTLVGALLRAATYYFCDLEIACVGEHKRVFWQPNGAPRTTQLGDNPMVFSHNKVTRFAIPYTAPHRLLSTVYNGECEYTKTVTAIRGDREVLAAKYSSAKHTLPSTFNFGFVTADEPVDVYYRMKRAEL

SEQ ID NO: 29 - DNA, 2181 bp, synthetic construct
  misc_feature 1..2181: note = nucleic acid, Consensus vp1-4 Subtype SAT3
  source 1..2181: mol_type = other DNA
atgctggacgtggactggcaggaccgcgccggcctgttcctgcgcggcgccggccagagcagccccgccaccggcagccagaaccagagcggcaacaccggcagcatcatcaacaactactacatgcagcagtaccagaacagcatggacacccagctgggcgacaacgccatcagcggcggcagcaacgagggcagcaccgacaccaccagcacccacaccaacaacacccagaacaacgactggttcagcaagctggcccagagcgccatcagcggcctgttcggcgccctgctggccgacaagaagaccgaggagaccacccacctggaggaccgcatcctgaccacccgccacaacaccaccaccagcaccacccagagcagcgtgggcgtgacctacggctacgtgagcgccgaccgcttcctgcccggccccaacaccagcggcctggagagccgcgtggagcaggccgagcgcttcttcaaggagaagctgttcacctggaccgccagccaggagtacgcccacgtgcacctgctggagctgcccaccgaccacaagggcatctacggcgccatggtggacagccacgcctacgtgcgcaacggctgggacgtgcaggtgaccgccaccagcacccagttcaacggcggcaccctgctggtggccatggtgcccgagctgcacagcctggacacccgcgacgtgagccagctgaccctgttcccccaccagttcatcaacccccgcaccaacaccaccgcccacatcgtggtgccctacgtgggcgtgaaccgccacgaccaggtgcagatgcacaaggcctggaccctggtggtggccgtgatggcccccctgaccaccagcagcatgggccaggacaacgtggaggtgtacgccaacatcgcccccaccaacgtgtacgtggccggcgagcgccccagcaagcagggcatcatccccgtggcctgcaacgacggctacggcggcttccagaacaccgaccccaagaccgccgaccccatctacggcctggtgagcaaccccccccgcaccgccttccccggccgcttcaccaacctgctggacgtggccgaggcctgccccaccttcctggacttcgacggcgtgccctacgtgaagaccacccacaacagcggcagcaagatcctgacccacatcgacctggccttcggccacaagagcttcaagaacacctacctggccggcctggcccagtactacgcccagtacagcggcagcatcaacctgcacttcatgtacaccggccccacccagagcaaggcccgcttcatggtggcctacatcccccccggcaccaccgtgcccaacacccccgagcaggccgcccactgctaccacagcgagtgggacaccggcctgaacagcaagttcaccttcaccgtgccctacatgagcgccgccgacttcgcctacacctactgcgacgagcccgagcaggccagcgcccagggctgggtgaccctgtaccagatcaccgacacccacgaccccgacagcgccgtgctggtgagcgtgagcgccggcgccgacttcgagctgcgcctgcccatcaaccccgccgcccagaccaccagcgccggcgagggcgccgacgtggtgaccaccgacgtgaccacccacggcggcgaggtgagcgtgccccgccgccagcacaccaacgtggagttcctgctggaccgcttcacccacatcggcaccatcaacggccaccgcaccatctgcctgatggacaccaaggagcacaccctggtgggcgccatcctgcgcagcgccacctactacttctgcgacctggaggtggccgtgctgggcaacgccaagtacgccgcctgggtgcccaacggctgcccccacaccgaccgcgtggaggacaaccccgtggtgcacagcaagggcagcgtggtgcgcttcgccctgccctacaccgccccccacggcgtgctggccaccgtgtacaacggcaactgcaagtacagcaccacccagcgcgtggccccccgccgcggcgacctgggcgtgctgagccagcgcgtggagaacgagaccacccgctgcatccccaccaccttcaacttcggccgcctgctgtgcgagagcggcgacgtgtactaccgcatgaagcgcaccgagctg

SEQ ID NO: 30 - protein, 727 aa, synthetic construct
  REGION 1..727: note = Consensus vp1-4 Subtype SAT3
MLDVDWQDRAGLFLRGAGQSSPATGSQNQSGNTGSIINNYYMQQYQNSMDTQLGDNAISGGSNEGSTDTTSTHTNNTQNNDWFSKLAQSAISGLFGALLADKKTEETTHLEDRILTTRHNTTTSTTQSSVGVTYGYVSADRFLPGPNTSGLESRVEQAERFFKEKLFTWTASQEYAHVHLLELPTDHKGIYGAMVDSHAYVRNGWDVQVTATSTQFNGGTLLVAMVPELHSLDTRDVSQLTLFPHQFINPRTNTTAHIVVPYVGVNRHDQVQMHKAWTLVVAVMAPLTTSSMGQDNVEVYANIAPTNVYVAGERPSKQGIIPVACNDGYGGFQNTDPKTADPIYGLVSNPPRTAFPGRFTNLLDVAEACPTFLDFDGVPYVKTTHNSGSKILTHIDLAFGHKSFKNTYLAGLAQYYAQYSGSINLHFMYTGPTQSKARFMVAYIPPGTTVPNTPEQAAHCYHSEWDTGLNSKFTFTVPYMSAADFAYTYCDEPEQASAQGWVTLYQITDTHDPDSAVLVSVSAGADFELRLPINPAAQTTSAGEGADVVTTDVTTHGGEVSVPRRQHTNVEFLLDRFTHIGTINGHRTICLMDTKEHTLVGAILRSATYYFCDLEVAVLGNAKYAAWVPNGCPHTDRVEDNPVVHSKGSVVRFALPYTAPHGVLATVYNGNCKYSTTQRVAPRRGDLGVLSQRVENETTRCIPTTFNFGRLLCESGDVYYRMKRTEL

SEQ ID NO: 31 - DNA, 678 bp, synthetic construct
  misc_feature 1..678: note = nucleic acid consensus VP1-Asia
  source 1..678: mol_type = other DNA
atggactggacctggatcctgttcctggtcgccgctgccactagggtgcacagcaccaccaccaccggcgagagcgccgaccccgtgaccaccaccgtggagaactacggcggcgagacacagaccgccaggcgcctccacaccgacgtggccttcgtgctggacagattcgtgaagctgacccagcccaagagcacccagaccctggacctgatgcagatccccagccacaccctcgtgggcgccctgctgagaagcgccacctactacttcagcgacctggaagtggccctggtgcacaccggccctgtgacctgggtgcccaacggcgctcccaagaccgccctgaacaaccacaccaaccccaccgcctaccagaagcagcccatcaccaggctggccctgccctacaccgcccctcacagggtgctgtccaccgtgtacaacggcaagaccacctacggcgaggaaagcagcagaaggggcgatctggccgctctcgccaggcgcgtgaacaacaggctgcccacctccttcaactatggcgccgtcaaggccgacaccatcaccgagctgctgatcaggatgaagagggccgagacatactgccccaggcccctgctggccctggacaccacccaggacaggcgcaagcaggaaatcattgcccccgagaag

SEQ ID NO: 32 - protein, 229 aa, synthetic construct
  REGION 1..229: note = Consensus VP1-Asia
MDWTWILFLVAAATRVHSTTTTGESADPVTTTVENYGGETQTARRLHTDVAFVLDRFVKLTQPKSTQTLDLMQIPSHTLVGALLRSATYYFSDLEVALVHTGPVTWVPNGAPKTALNNHTNPTAYQKQPITRLALPYTAPHRVLSTVYNGKTTYGEESSRRGDLAALARRVNNRLPTSFNYGAVKADTITELLIRMKRAETYCPRPLLALDTTQDRRKQEIIAPEKQTL

SEQ ID NO: 33 - DNA, 742 bp, synthetic construct
  misc_feature 1..742: note = nucleic acid consensus VP1-O
  source 1..742: mol_type = other DNA
atggactggacctggatcctgttcctggtcgccgctgccactagggtgcacagcaccacctctaccggggagtccgccgatcctgtgacagccacagtggaaaattacggcggggaaacccaggtgcagaggcggcagcacaccgatgtgtctttcatcctggaccgcttcgtgaaagtgacccccaaggccgaccagatcaacgtgctggatctcatgcagattcccgcccatacactcgtcggggctctgctgcgcaccgccacatactatttcgccgatctcgaggtggccgtgaagcacgagggcaacctgacatgggtgccaaatggcgcccctgaggccgctctggacaacaccaccaatcctacagcctaccacaaggcccccctgaccagactggctctgccttatacagccccccaccgcgtgctggccacagtgtataatggcaactgcaagtacggcgaagtggccgtcaccaacgtgcgcggcgacctccaggtgctggcccagaaggccgccaggaccctgcctaccagctttaactacggggccatcaaggccaccagagtgaccgaactgctgtacagaatgaagcgcgccgaaacctactgccctagacctctgctcgccatccaccccagcgaggccaggcacaagcagaaaattgtggcccctgtgaagcagctgctgtgatgactgcagatatccagcacagtggcggccgctcgagtctaga

SEQ ID NO: 34 - protein, 201 aa, synthetic construct
  REGION 1..201: note = Consensus VP1- O
MDWTWILFLVAAATRVHSTTSTGESADPVTATVENYGGETQVQRRQHTDVSFILDRFVKVTPKADQINVLDLMQIPAHTLVGALLRTATYYFADLEVAVKHEGNLTWVPNGAPEAALDNTTNPTAYHKAPLTRLALPYTAPHRVLATVYNGNCKYGEVAVTNVRGDLQVLAQKAARTLPTSFNYGAIKATRVTELLYRMKR

SEQ ID NO: 35 - DNA, 771 bp, synthetic construct
  misc_feature 1..771: note = nucleic acid consensus VP1-A
  source 1..771: mol_type = other DNA
atggactggacctggattctgtttctggtggccgctgccacaagagtgcactccaccacctctgccggcgagtccgccgacccagtgaccaccaccgtggagaactacggcggcgagacacaggtgcagcgcaggcaccacaccgacgtgggcttcatcatggaccgcttcgtgaagatcggcaacacctcccccacccacgtgatcgacctgatgcagacccaccagcacggactggtgggagccctgctgagagccgccacctactacttctccgacctggaaatcgtggtgcgccacgacggaaacctgacatgggtgcccaatggcgccccagaggccgccctgtccaacaccggcaaccccaccgcctacaacaaggcccccttcaccagactggccctgccatacaccgcccctcacagggtgctggccaccgtgtacaacggcaccaacaagtactccgccgcctccggaagaacaagaggcgacctgggcaccgtggccgccagaatcgccgcccagctgcccgcctccttcaacttcggcgccatcaaggccgacgccatccacgaactgctggtgcgcatgaagcgcgccgagctgtactgcccaagacccctgctggccgtggaggtgtcctcccaggaccgccacaagcagaagatcatcgccccagccaagcagctgctgtacccctacgacgtgcccgactacgcctccctgggcggaccatgatgactcgagtctagagggcccgtttaaacccgc

SEQ ID NO: 36 - protein, 231 aa, synthetic construct
  REGION 1..231: note = Consensus VP1-A
MDWTWILFLVAAATRVHSTTSAGESADPVTTTVENYGGETQVQRRHHTDVGFIMDRFVKIGNTSPTHVIDLMQTHQHGLVGALLRAATYYFSDLEIVVRHDGNLTWVPNGAPEAALSNTGNPTAYNKAPFTRLALPYTAPHRVLATVYNGTNKYSAASGRTRGDLGTVAARIAAQLPASFNFGAIKADAIHELLVRMKRAELYCPRPLLAVEVSSQDRHKQKIIAPAKQLL

SEQ ID NO: 37 - DNA, 732 bp, synthetic construct
  misc_feature 1..732: note = nucleic acid consensus VP1-C
  source 1..732: mol_type = other DNA
atggactggacctggattctgtttctcgtggccgctgccacaagagtgcacagcaccaccaccaccggcgagtccgccgacccagtgaccaccaccgtggagaactacggcggcgagacacagacccagcgcaggcaccacacagacgtggccttcgtgctggaccgcttcgtgaaggtgcaggtgtccggcaaccagcacaccctggacgtgatgcaggtgcacaaggactccatcgtgggcgccctgctgagagccgccacctactacttctccgacctggaaatcgccgtgacccacaccggaaagctgacctgggtgcccaatggcgccccagtgtccgccctggacaacaccaccaaccccaccgcctaccacaagggcccactgaccagactggccctgccatacaccgcccctcacagagtgctggccacagcctacaccggcacaaccgcctactccgcctccgccagaagaggcgatctggcccacctcgccgctgcccacgccagacacctgcccaccagcttcaacttcggcgccgtgaaggccgagacaatcaccgagctgctggtgcgcatgaagcgcgccgagctgtactgccccagacccgtgctgccagtgcagccatccggcgacagacacaagcagcccctgatcgccccagccaagcagctgctgtacccctacgacgtgcccgactacgcctccctgggcggaccatgatga

SEQ ID NO: 38 - protein, 228 aa, synthetic construct
  REGION 1..228: note = Consensus VP1-C
MDWTWILFLVAAATRVHSTTTTGESADPVTTTVENYGGETQTQRRHHTDVAFVLDRFVKVQVSGNQHTLDVMQVHKDSIVGALLRAATYYFSDLEIAVTHTGKLTWVPNGAPVSALDNTTNPTAYHKGPLTRLALPYTAPHRVLATAYTGTTAYSASARRGDLAHLAAAHARHLPTSFNFGAVKAETITELLVRMKRAELYCPRPVLPVQPSGDRHKQPLIAPAKQLL

SEQ ID NO: 39 - DNA, 1404 bp, synthetic construct
  misc_feature 1..1404: note = nucleic acid, VP1-A+ VP1-C sequence
  source 1..1404: mol_type = other DNA
atggactggacctggattctgtttctcgtggccgctgctacaagagtgcactccaccacctctgccggcgagtccgccgacccagtgaccaccaccgtggagaactacggcggcgagacacaggtgcagcgcaggcaccacaccgacgtgggcttcatcatggaccgcttcgtgaagatcggcaacacctcccccacccacgtgatcgacctgatgcagacccaccagcacggactggtgggagccctgctgagagccgccacctactacttctccgacctggaaatcgtggtgcgccacgacggaaacctgacctgggtgcccaatggcgctccagaggccgccctgtccaacaccggcaaccccaccgcctacaacaaggcccccttcaccagactggccctgccatacaccgcccctcacagggtgctggccaccgtgtacaacggcaccaacaagtactccgccgcctccggaagaacaagaggcgacctgggcaccgtggccgccagaatcgccgcccagctgcccgcctccttcaacttcggcgccatcaaggccgacgccatccacgaactgctggtgcgcatgaagcgcgccgagctgtactgcccaagacccctgctggccgtggaggtgtcctcccaggaccgccacaagcagaagatcatcgccccagccaagcagctgctgcgcggcaggaagagaagatccaccaccaccacaggcgaaagcgccgatcccgtgacaacaacagtggaaaattacggcggggaaacccagacccagcggcgccaccacacagatgtggccttcgtgctggacagattcgtgaaggtgcaggtgtccggcaaccagcacaccctggacgtgatgcaggtgcacaaggactccatcgtgggcgccctgctgagggccgccacctactactttagcgatctggaaatcgccgtgacccacaccggcaagctgacatgggtgccaaatggggcccctgtgtctgccctggacaacaccaccaacccaacagcctaccacaaagggccactgacacgcctggccctgccttacacagccccacaccgcgtgctggccacagcctacaccggaaccaccgcctactccgcctccgccagaagaggcgatctggcccatctggccgctgcccacgccagacacctgcccaccagcttcaactttggggccgtgaaagccgagacaatcaccgagctgctggtgcggatgaagagggccgaactgtactgtcctcgccccgtgctgccagtgcagccatccggcgacagacacaagcagcccctgatcgcccctgccaaacagctgctgtacccctacgacgtgcccgactacgcctccctgggcggaccatgatgactcgagtctaga

SEQ ID NO: 40 - protein, 448 aa, synthetic construct
  REGION 1..448: note = VP1-A+ VP1-C amino acid sequence
MDWTWILFLVAAATRVHSTTSAGESADPVTTTVENYGGETQVQRRHHTDVGFIMDRFVKIGNTSPTHVIDLMQTHQHGLVGALLRAATYYFSDLEIVVRHDGNLTWVPNGAPEAALSNTGNPTAYNKAPFTRLALPYTAPHRVLATVYNGTNKYSAASGRTRGDLGTVAARIAAQLPASFNFGAIKADAIHELLVRMKRAELYCPRPLLAVEVSSQDRHKQKIIAPAKQLLRGRKRRSTTATGESADPVTTTVENYGGETQTQRRHHTDVAFVLDRFVKVQVSGNQHTLDVMQVHKDSIVGALLRAATYYFSDLEIAVTHTGKLTWVPNGAPVSALDNTTNPTAYHKGPLTRLALPYTAPHRVLATAYTGTTAYSASARRGDLAHLAAAHARHLPTSFNFGAVKAETITELLVRMKRAELYCPRPVLPVQPSGDRHKQPLIAPAKQLL

SEQ ID NO: 41 - DNA, 1350 bp, synthetic construct
  misc_feature 1..1350: note = nucleic acid VP1-Asia + VP1-O sequence
  source 1..1350: mol_type = other DNA
atggactggacctggatcctgttcctggtcgccgctgccactagggtgcacagcaccaccaccaccggcgagagcgccgaccccgtgaccaccaccgtggagaactacggcggcgagacacagaccgccaggcgcctccacaccgacgtggccttcgtgctggacagattcgtgaagctgacccagcccaagagcacccagaccctggacctgatgcagatccccagccacaccctcgtgggcgccctgctgagaagcgccacctactacttcagcgacctggaagtggccctggtgcacaccggccctgtgacctgggtgcccaacggcgctcccaagaccgccctgaacaaccacaccaaccccaccgcctaccagaagcagcccatcaccaggctggccctgccctacaccgcccctcacagggtgctgtccaccgtgtacaacggcaagaccacctacggcgaggaaagcagcagaaggggcgatctggccgctctcgccaggcgcgtgaacaacaggctgcccacctccttcaactatggcgccgtcaaggccgacaccatcaccgagctgctgatcaggatgaagagggccgagacatactgccccaggcccctgctggccctggacaccacccaggacaggcgcaagcaggaaatcattgcccccgagaagcagaccctgaggggcaggaagaggcgctccaccacctctaccggggagtccgccgatcctgtgacagccacagtggaaaattacggcggggaaacccaggtgcagaggcggcagcacaccgatgtgtctttcatcctggaccgcttcgtgaaagtgacccccaaggccgaccagatcaacgtgctggatctcatgcagattcccgcccatacactcgtcggggctctgctgcgcaccgccacatactatttcgccgatctcgaggtggccgtgaagcacgagggcaacctgacatgggtgccaaatggcgcccctgaggccgctctggacaacaccaccaatcctacagcctaccacaaggcccccctgaccagactggctctgccttatacagccccccaccgcgtgctggccacagtgtataatggcaactgcaagtacggcgaagtggccgtcaccaacgtgcgcggcgacctccaggtgctggcccagaaggccgccaggaccctgcctaccagctttaactacggggccatcaaggccaccagagtgaccgaactgctgtacagaatgaagcgcgccgaaacctactgccctagacctctgctcgccatccacccagcgaggccaggcacaagcagaaaattgtggcccctgtgaagcagctgctgt

SEQ ID NO: 42 - protein, 470 aa, synthetic construct
  REGION 1..470: note = VP1-Asia + VP1-O amino acid sequence
MDWTWILFLVAAATRVHSTTTTGESADPVTTTVENYGGETQTARRLHTDVAFVLDRFVKLTQPKSTQTLDLMQIPSHTLVGALLRSATYYFSDLEVALVHTGPVTWVPNGAPKTALNNHTNPTAYWVPNGAPKTALNNHTNPTAYQKQPITRLALPYTAPHRVLSTVYNGKTTYGEESSRRGDLAALARRVNNRLPTSFNYGAVKADTITELLIRMKRAETYCPRPLLALDTTQDRRKQEIIAPEKQTLRGRKRRSTTSTGESADPVTATVENYGGETQVQRRQHTDVSFILDRFVKVTPKADQINVLDLMQIPAHTLVGALLRTATYYFADLEVAVKHEGNLTWVPNGAPEAALDNTTNPTAYHKAPLTRLALPYTAPHRVLATVYNGNCKYGEVAVTNVRGDLQVLAQKAARTLPTSFNYGAIKATRVTELLYRMKRAETYCPRPLLAIHPSEARHKQKIVAPVKQLL

SEQ ID NO: 43 - DNA, 56 bp, synthetic construct
  misc_feature 1..56: note = IgE leader sequence
  source 1..56: mol_type = other DNA
atggactggacctggatcctgttcctggtcgccgctgccactagggtgcacagcac

SEQ ID NO: 44 - protein, 18 aa, synthetic construct
  REGION 1..18: note = IgE amino acid sequence
MDWTWILFLVAAATRVHS

SEQ ID NO: 45 - protein, 7 aa, synthetic construct
  REGION 1..7: note = amino acid sequence 1
RGRKRRS

SEQ ID NO: 46 - protein, 229 aa, synthetic construct; identical to SEQ ID NO: 32
  REGION 1..229: note = amino acid sequence with Type Asia 1 VP1

SEQ ID NO: 47 - protein, 232 aa, synthetic construct
  REGION 1..232: note = amino acid sequence with Type O VP1
MDWTWILFLVAAATRVHSTTSTGESADPVTATVENYGGETQVQRRQHTDVSFILDRFVKVTPKADQINVLDLMQIPAHTLVGALLRTATYYFADLEVAVKHEGNLTWVPNGAPEAALDNTTNPTAYHKAPLTRLALPYTAPHRVLATVYNGNCKYGEVAVTNVRGDLQVLAQKAARTLPTSFNYGAIKATRVTELLYRMKRAETYCPRPLLAIHPSEARHKQKIVAPVKQLL

SEQ ID NO: 48 - protein, 231 aa, synthetic construct; identical to SEQ ID NO: 36
  REGION 1..231: note = amino acid sequence with Type A VP1

SEQ ID NO: 49 - protein, 228 aa, synthetic construct
  REGION 1..228: note = amino acid sequence with Type C VP1
MDWTWILFLVAAATRVHSTTATGESADPVTTTVENYGGETQTQRRHHTDVAFVLDRFVKVQVSGNQHTLDVMQVHKDSIVGALLRAATYYFSDLEIAVTHTGKLTWVPNGAPVSALDNTTNPTAYHKGPLTRLALPYTAPHRVLATAYTGTTAYSASARRGDLAHLAAAHARHLPTSFNFGAVKAETITELLVRMKRAELYCPRPVLPVQPSGDRHKQPLIAPAKQLL